(19) United States
(12) Patent Application Publication
(10) Pub. No.: US 2017/0226483 A1
(43) Pub. Date: Aug. 10, 2017

(54) Methods and Vectors to Produce Vector Free Induced Pluripotent Stem Cells

(71) Applicant: Lonza Walkersville, Inc., Walkersville, MD (US)

(72) Inventors: Eytan Abraham, Potomac, MD (US); Thomas Payne, Cambridge (GB); Robert J. Young, London (GB); Inbar Friedrich Ben Nun, Rockville, MD (US)

(73) Assignee: Lonza Walkersville, Inc., Walkersville, MD (US)

(21) Appl. No.: 15/404,960

(22) Filed: Jan. 12, 2017

Related U.S. Application Data
(60) Provisional application No. 62/277,784, filed on Jan. 12, 2016.

(51) Int. Cl. C12N 5/074; C07K 14/47; C12N 15/85; C07K 14/005; C12N 9/12; C12N 9/78

(52) U.S. Cl. CPC C12N 5/0696; C12Y 207/01021; C12N 9/1211; C12Y 305/04001; C12N 9/78; C12N 15/85; C07K 14/005; C07K 14/4702; C12N 2820/60; C12N 2830/003; C12N 2830/006; C12N 2510/00; C12N 2506/11; C12N 2800/108; C12N 2710/16231

(57) ABSTRACT

The invention relates generally to methods of generating induced pluripotent stem cells (iPSCs) that do not contain the reprogramming vector. In some embodiments, the invention relates to inducing pluripotency in somatic cells by introducing an episomal vector(s) comprising at least one expression cassette containing reprogramming factors and/or synthetic transcription factors and a suicide gene. In some embodiments, the invention relates to inducing pluripotency in somatic cells by introducing episomal vector(s) comprising expression cassettes containing reprogramming factors and/or synthetic transcription factors and a transcriptionally regulated EBNA-1 gene. In some embodiments, the invention relates to inducing pluripotency in somatic cells by introducing episomal vector(s) comprising expression cassettes containing reprogramming factors and/or synthetic transcription factors and both a suicide gene and a transcriptionally regulated EBNA-1 gene.

### BACKGROUND OF THE INVENTION

Field of the Invention

The invention relates generally to methods of generating induced pluripotent stem cells (iPSCs) that do not contain a reprogramming vector. In some embodiments, the invention relates to inducing pluripotency in somatic cells by introducing an episomal vector(s) comprising at least one expression cassette containing reprogramming factors and/or synthetic transcription factors and a suicide gene.

### BACKGROUND

Current cellular reprogramming methods producing induced pluripotent stem cells (iPSCs) often use retroviruses to deliver reprogramming factors. (Schlaeger et al., “A comparison of non-integrating reprogramming methods,” Nat Biotechnol 33(1): 58-63 (2015) (“Schlaeger”)). RNA from these viruses is reverse transcribed into DNA and integrated into the genome of the host cell. However, such cells are not acceptable under regulatory guidelines for therapeutic applications. Consequently other reprogramming methods are needed to ensure that iPSCs are free of any exogenous DNA sequences.

As summarized by Schlaeger, major approaches include use of messenger RNA transfection, with and without micro-RNAs, and episomal, EBNA-1, based expression vectors. The former, RNA-based, methods can efficiently generate iPSCs, but present significant issues relating to labor requirements and success rates (method robustness), as well as cell type and donor specificity.

Episomal vector reprogramming represents an attractive ‘all-round’ system for reprogramming, taking into account all of the required characteristics of the method. A major problem remains, however, that extensive passaging is required before exogenous DNA-free lines are established, either due to prolonged vector retention or integration into the host cell genome.

Current cellular reprogramming protocols using episomal vectors require that quantitative measures are taken to ensure that the generated iPSC lines are vector-free. Newly generated iPSCs are typically picked from the P0 plate on day 20-30 after transfection. Most of the colonies retain the vector when picked, and vector-loss kinetics varies in different iPSC clones, with some clones identified as vector-free at low passage (5-10) and other at a high passage (15-30). This makes it necessary to pick many iPSC colonies from the P0 plate and to keep them in culture for a long period of time until vector clearance is achieved. This practice is a hurdle for cell therapy applications, where time and cost are crucial factors.

### SUMMARY OF THE INVENTION

In some embodiments, the invention provides efficient methods of producing induced human pluripotent stem cells (iPSCs) that are essentially free of reprogramming vector(s), comprising: (a) introducing the reprogramming vector(s) into a human somatic cell to produce a first cell population, wherein the reprogramming vector(s) comprises a viral origin of replication, an expression cassette encoding at least one iPSC reprogramming factor and/or synthetic transcription factor, and a suicide gene; (b) culturing the first cell population to effect expression of the reprogramming factor(s) and/or synthetic transcription factor to produce a second cell population having traits consistent with embryonic stem cells; and (c) contacting the second cell population with a suicide gene substrate to produce a cell population that is essentially free of reprogramming vector(s).

In some embodiments, the invention is directed to methods of producing induced human pluripotent stem cells (iPSCs) that are essentially free of episomal reprogramming vector(s), comprising: (a) introducing an episomal reprogramming vector(s) into a somatic cell to produce a first cell population, wherein the episomal reprogramming vector(s) comprises (i) an OriP replication origin, (ii) an expression cassette encoding an iPSC reprogramming factor and/or synthetic transcription factor, (iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1, and (iv) a thymidine kinase or cytosine deaminase suicide gene; (b) culturing the first cell population to effect expression of the reprogramming factor and/or synthetic transcription factor to produce a second cell population having traits consistent with embryonic stem cells; (c) contacting the second cell population with a suicide gene substrate to produce iPSCs that are essentially free of an episomal reprogramming vector.

In additional embodiments, the invention provides methods of producing induced human pluripotent stem cells (iPSCs) that are essentially free of reprogramming vector(s), comprising: (a) introducing a reprogramming vector(s) into a somatic cell to produce a first cell population, wherein the reprogramming vector comprises i) a viral origin of replication, ii) an expression cassette encoding iPSC reprogramming factor(s) and/or synthetic transcription factor(s), iii) a gene regulating extrachromosomal replication and partitioning of the reprogramming vector, and iv) a regulated promoter system; (b) culturing the first cell population to effect expression of the reprogramming factors and/or synthetic transcription factors to produce a second cell population having traits consistent with embryonic stem cells, wherein during culture of the first cell population the reprogramming vector is replicated; and (c) culturing the second cell population wherein the gene regulating extrachromosomal replication and partitioning of the reprogramming vector is regulated such that the reprogramming vector is lost during cell division, to produce iPSCs that are essentially free of the reprogramming vector.

In additional embodiments, the invention provides methods of producing induced human pluripotent stem cells (iPSCs) that are essentially free of episomal reprogramming vector(s), comprising: (a) introducing episomal reprogramming vector(s) into a somatic cell to produce a first cell population, wherein the episomal reprogramming vector comprises (i) an OriP replication origin, (ii) an expression cassette encoding an iPSC reprogramming factor and/or synthetic transcription factor, iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1, and (iv) a tetracycline or tetracycline derivative regulated promoter system (TetOn or TetOff); (b) culturing the first cell population to produce a second cell population having traits consistent with embryonic stem cells, wherein during culturing the episomal reprogramming vector is replicated; (c) culturing the second cell population to produce a third cell population, wherein during culturing the episomal reprogramming vector is not replicated; (d) selecting colonies from the third cell population to produce a fourth cell population; and (e) culturing the fourth cell population to produce iPSCs that are essentially free of the episomal reprogramming vector.

In additional embodiments, the invention provides methods of producing induced human pluripotent stem cells (iPSCs) that are essentially free of reprogramming vector(s), comprising: (a) introducing a reprogramming vector(s) into a somatic cell to produce a first cell population, wherein the reprogramming vector comprises i) a viral origin of replication, ii) an expression cassette encoding iPSC reprogramming factor(s) and/or synthetic transcription factor(s), iii) a gene regulating extrachromosomal replication and partitioning of the reprogramming vector, iv) a regulated promoter system, and v) a suicide gene; (b) culturing the first cell population to effect expression of the reprogramming factor and/or synthetic transcription factor to produce a second cell population having traits consistent with embryonic stem cells, wherein during culture the reprogramming vector is replicated; (c) culturing the second cell population wherein the gene regulating extrachromosomal replication and partitioning is regulated such that the reprogramming vector is lost during cell division, to produce a third cell population comprising iPSCs that are substantially free of the reprogramming factor, and (d) contacting the third cell population with a suicide gene substrate to produce iPSCs that are essentially free of the reprogramming vector.

In additional embodiments, the invention provides methods of producing induced human pluripotent stem cells (iPSCs) that are essentially free of episomal reprogramming vector(s), comprising: (a) introducing episomal reprogramming vector(s) into a somatic cell to produce a first cell population, wherein the episomal reprogramming vector comprises (i) an OriP replication origin, (ii) an expression cassette encoding an iPSC reprogramming factor and/or synthetic transcription factor, iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1, (iv) a tetracycline or tetracycline derivative regulated promoter system (TetOn or TetOff), and v) a thymidine kinase or cytosine deaminase suicide gene; (b) culturing the first cell population to effect expression of the reprogramming factors to produce a second cell population having traits consistent with embryonic stem cells, wherein during culturing the episomal reprogramming vector is replicated; (c) culturing the second cell population to produce a third cell population comprising iPSCs that are substantially free of the episomal reprogramming vector, wherein during culturing of the second cell population, the episomal reprogramming vector is not replicated; and (d) contacting the third cell population with a suicide gene substrate to produce iPSCs that are essentially free of the episomal reprogramming vector.

In embodiments, the invention provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a suicide gene.

In additional embodiments, the invention provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a thymidine kinase or cytosine deaminase suicide gene.

The invention further provides, in embodiments, an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a regulated promoter system.

In embodiments, the invention additionally provides an episomal reprogramming vector comprising: (a) an OriP origin of replication; (b) an expression cassette encoding an iPSC reprogramming factor and/or synthetic transcription factor, (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a TetOn or TetOff system.

In embodiments, the invention additionally provides an episomal reprogramming vector comprising: (a) an OriP origin of replication; (b) an expression cassette encoding an iPSC reprogramming factor and/or synthetic transcription factor, (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; (d) a TetOn or TetOff system; and (e) a suicide gene.

In embodiments, the invention additionally provides an episomal reprogramming vector comprising: (a) an OriP origin of replication; (b) an expression cassette encoding an iPSC reprogramming factor and/or synthetic transcription factor, (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; (d) a TetOn or TetOff system; and (e) a thymidine kinase or cytosine deaminase suicide gene.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts the procedure of iPSC colonies expansion and analysis.

### DETAILED DESCRIPTION OF THE INVENTION

### Definitions

Unless defined otherwise, all technical and scientific terms used herein have their common meaning as understood by one of ordinary skill in the art to which this invention is related.

Before describing the present invention in detail, it is to be understood that this invention is not limited to specific compositions or process steps, as such can vary. As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A,” (alone) and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or embodiments of the invention, which can be had by reference to the specification as a whole. Accordingly, the terms defined immediately below are more fully defined by reference to the specification in its entirety.

It is understood that wherever embodiments are described herein with the language “comprising,” otherwise analogous embodiments described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

“Reprogramming” is the conversion of one specific cell type to another. For example, regramming is the conversion of a somatic cell type, such as a fibroblast, to a pluripotent cell type. A cell is reprogrammed if, after sufficient proliferation, a measurable proportion of cells subjected to reprogramming (progeny) have phenotypic characteristics of a new cell type than before reprogramming. Under certain conditions, the proportion of progeny with characteristics of the new cell type may be at least about 0.05%, 0.1%, 0.2%, 0.5%, 1%, 5%, 25% or more.

A “vector” or “construct” (sometimes referred to as gene delivery or gene transfer “vehicle”) refers to a macromolecule or complex of molecules comprising a polynucleotide to be delivered to a host cell, either in vitro or in vivo. A vector can be a linear or a circular molecule.

“Episomal vector” refers to a vector (defined above) that replicates independently of the chromosomal DNA in the cell where the vector resides.

A “plasmid”, a common type of vector, is an extra-chromosomal DNA molecule separate from the chromosomal DNA which capable of replicating independently of the chromosomal DNA in a compatible cell. For example, certain plasmids, such as pUC, replicate independently in bacteria, but do not do so in mammalian cells. In certain cases, it is circular and double-stranded.

An “origin of replication” (“ori”) or “replication origin” is a DNA sequence involved in DNA replication. Commonly these are derived from bacteria or viruses, each with distinct features. A bacterial ‘ori’ is required for DNA replication in bacterial cells. All common plasmids/vectors will contain such a region (often derived from pUC vectors) to allow efficient propagation. A viral ‘ori’, e.g., from a lymphotrophic herpes virus, functions to allow replication in mammalian cells. When present, and with presence of the appropriate tethering protein (e.g. EBNA-1), a cell is capable of maintaining the vector at a site at or near where DNA synthesis initiates, resulting in DNA replication and partitioning during cell division. An ori for EBV includes FR sequences (20 imperfect copies of a 30 bp repeat), and preferably DS sequences. However, other sites in EBV bind EBNA-1, e.g., Rep* sequences can substitute for DS as an origin of replication. Thus, a replication origin of EBV includes FR, DS or Rep* sequences or any functionally equivalent sequences through nucleic acid modifications or synthetic combination derived therefrom. For example, the present invention may also use genetically engineered replication origin of EBV, such as by insertion or mutation of individual elements.

The term “OriP” refers to the region of the Epstein-Barr virus chromosome that supports the replication and stable maintenance of plasmids in human cells.

A “lymphotrophic” herpes virus is a herpes virus that replicates in a lymphoblast (e.g., a human B lymphoblast) or other cell types and replicates extra-chromosomally for at least a part of its natural life-cycle. After infecting a host, these viruses latently infect the host by maintaining the viral genome as a plasmid. Exemplary lymphotropic herpes viruses include, but are not limited to Epstein Barr virus (EBV), Kaposi's sarcoma herpes virus (KSHV), Herpes virus saimiri (HS) and Marek's disease virus (MDV).

A “template” as used herein is a DNA molecule which is specifically bound by a wild-type protein of a lymphotrophic herpes virus, which wild-type protein corresponds to EBNA-1, as a result of the presence in that template of a DNA sequence which is bound by the wild-type protein with an affinity that is at least 10% that of the binding of a DNA sequence corresponding to OriP of EBV by the wild-type protein and from which template transcription is optionally initiated and/or enhanced after the protein binds and/or the maintenance of which template in a cell is enhanced. An “integrated template” is one which is stably maintained in the genome of the cell, e.g., integrated into a chromosome of that cell. An “extra-chromosomal template” is one which is maintained stably maintained in a cell but which is not integrated into the chromosome.

The term “control elements” refers collectively to promoter regions, polyadenylation signals, transcription termination sequences, upstream regulatory domains, origins of replication, internal ribosome entry sites (“IRES”), enhancers, splice junctions, and the like, which collectively provide for the replication, transcription, post-transcriptional processing and translation of a coding sequence in a recipient cell. Not all of these control elements need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′ direction) coding sequence.

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified. Examples of somatic cells include mononuclear cells, such as peripheral blood mononuclear cells, fibroblasts, keratinocytes, hematopoitic cells, mesenchymal cells, liver cells, stomach cells and β cells

Cells are “substantially free” of episomal reprogramming vectors and exogenous genetic elements (e.g., substantially free of reprogramming vector genetic elements), as used herein, when they have less that 10% of the element(s), and are “essentially free” of episomal reprogramming vectors and exogenous genetic elements (e.g., essentially free of reprogramming vector genetic elements) when they have less than 1% of the element(s). However, even more desirable are cell populations wherein less than 0.5% or less than 0.1% of the total cell population comprise exogenous genetic elements. Thus, iPS cell populations wherein less than 0.1% to 10% (including all intermediate percentages) of the cells of the population comprises undesirable exogenous genetic elements.

By “enhancer” is meant a nucleic acid sequence that, when positioned proximate to a promoter, confers increased transcription activity relative to the transcription activity resulting from the promoter in the absence of the enhancer domain.

By “expression construct” or “expression cassette” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means. In relationship to a cell, the term “exogenous” refers to a cell that was isolated and subsequently introduced to other cells or into an organism by artificial or natural means. An exogenous nucleic acid may be from a different organism or cell, or it may be one or more additional copies of a nucleic acid which occurs naturally within the organism or cell. An exogenous cell may be from a different organism, or it may be from the same organism. By way of a non-limiting example, an exogenous nucleic acid is in a chromosomal location different from that of natural cells, or is otherwise flanked by a different nucleic acid sequence than that found in nature.

The term “passaging” refers to the process of subculturing cells by transferring some or all of the cells from a previous culture to a new substrate, e.g., a new vessel, with fresh growth medium.

The term “suicide gene” is a nucleotide encoding a protein that, converts a non-toxic compound to a toxic form. Examples of suicide genes include the herpes simplex virus thymidine kinase/ganciclovir system, the cytosine deaminase/5-FU system, and the carboxyl esterase/irinotecan system. Often suicide genes are constitutively expressed, such that cellular toxicity results when the appropriate substrate is provided. The “suicide gene substrate” is the non-toxic compound that the suicide gene converts into a toxic form.

The term “corresponds to” means that a polynucleotide sequence is homologous (i.e., is identical, not strictly evolutionarily related) to all or a portion of a reference polynucleotide sequence, or that a polypeptide sequence is identical to a reference polypeptide sequence. The term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

A “gene,” “polynucleotide,” “coding region,” “sequence,” “segment,” “fragment,” or “transgene” that “encodes” a particular protein, is a nucleic acid molecule which is transcribed and optionally also translated into a gone product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self replicating, and has genetic information and a mechanism for expressing it. Cells used herein may be naturally-occurring cells or artificially modified cells (e.g., fusion cells, genetically modified cells, etc.).

The term “cell population” as used herein encompasses a group of clonal cells.

As used herein, the term “stem cell” refers to a cell capable of self replication and pluripotency. Typically, stem cells can regenerate an injured tissue. Stem cells herein may be, but are not limited to, embryonic stem (ES) cells or tissue stem cells (also called tissue-specific stem cell, or somatic stem cell). Any artificially produced cell which can have the above-described abilities (e.g., fusion cells, reprogrammed cells, or the like used herein) may be a stem cell.

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, are cells that have been artificially genetically reprogrammed to an embryonic stem cell-like state by expressing genes and factors for maintaining the defining properties of embryonic stem cells. iPSCs are derived from non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors. Synthetic transcription factors are non-naturally occurring reprogramming factors that can be introduced into somatic cells to reprogram the cells into an embryonic stem cell-like state.

“Pluripotency” refers to the ability of a cell to differentiate into cells derived from any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

“Homology” refers to the percent of identity between two polynucleotides or two polypeptides. The correspondence between one sequence and to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single strand-specific nuclease(s), and size determination of the digested fragments. Two DNA, or two polypeptide, sequences are “substantially homologous” to each other when at least about 80%, particularly at least about 90%, and most particularly at least about 95% of the nucleotides, or amino acids, respectively match over a defined length of the molecules, as determined using the methods above.

Overview

In the process of cellular reprogramming by non-integrating episomal vectors, quantitative measures typically have been necessary to ensure that the iPSC lines are vector-free. Current methods for generating vector-free iPSCs, include using DNA-mediated reprogramming, and involves picking multiple numbers of iPSC colonies from a P0 plate and expanding them to a point that there are enough cells to apply quantitative vector detection followed by further cultivation until the vector is cleared. While vector-free colonies could potentially be identified at passage 3, most of the iPSC clones retain the vector at passage 10 and higher. Cultivation for this length of time has serious implications on labor requirements, timelines and cost when carried out as part of an industrial process. In embodiments, the invention provides two different approaches to promote more rapid vector clearance: i) temporal regulation of vector retention post-reprogramming, and ii) incorporation of a suicide gene on reprogramming vector(s) to select for growth of vector-free colonies.

In embodiments, the present invention provides methods of generating exogenous DNA-free iPSCs for cell therapy by modifying the vectors, including episomal vectors that are used to deliver the reprogramming factors and/or synthetic transcription factors by (1) adding a suicide gene to the episomal vector, or (2) replacing constitutive EBNA-1 expression cassettes present on vectors with a regulated expression cassette on one or more vectors, where EBNA-1 expression is controlled via a regulated promoter system. These approaches could be used individually or in combination.

In embodiments, the invention provides numerous advantages, including (1) reducing the number of culture passages required before identifying vector-free iPSCs, (2) reducing the number of colonies needed to be picked and maintained before identifying vector-free iPSCs, (3) the ability to harvest iPSC colonies as a “pool”, instead of manually picking individual colonies, and (4) overall labor, time and cost savings.

Suicide Genes

Suicide genes are responsible for the conversion of non-toxic compounds to toxic forms. They can therefore be placed on expression vectors to serve as a negative selection for vector retention by adding the non-toxic suicide gene substrate at the appropriate time. Potential suicide genes which could be added to the episomal vector(s) include thymidine kinase and cytosine deaminase. The expression of these genes would be controlled by a constitutive promoter. Cell death would be induced only after adding their respective substrates, Ganciclovir (GNC) or 5-Flurocytosine (5-FC), directly to the culture medium.

Provision of the suicide gone substrate post-reprogramming (before colony picking or shortly after) will lead to cell death of iPSC colonies that retain the vector(s). Live colonies are those that have lost the vector(s) and therefore can be further picked and expanded. A screening assay provides an indication as to which iPSC colonies are promising for future expansion, banking and differentiation, without investing resources on expansion of non-vector-free iPSCs. Likewise, applying this strategy to select for iPSC colonies that are vector-free, enables harvesting of iPSCs as a “pool” without the need for manual picking of single colonies, and reduces the lag phase of cultivation before a robust cell line is established. Harvesting a pool of iPSCs shortens the time to achieve full characterization and banking, and additionally provides a diverse iPSC pool that increases the probability of efficient cell differentiation for downstream cell therapy applications.

Vector clearance kinetics may dictate colony picking even with the suicide gene approach, in the case that emerged iPSCs in the P0 plate still retain the vector at the time of adding the suicide gene substrate. Nevertheless, the suicide gene substrate could be provided in a ‘replica’ plate after picking, at P1 for example, to identify the vector-free iPSC clones without the need to carry out resource intensive qPCR screening.

The invention therefore provides methods of of producing induced human pluripotent stem cells (iPSCs) that are essentially free of reprogramming vector(s), comprising: (a) introducing the reprogramming vector(s) into a human somatic cell to produce a first cell population, wherein the reprogramming vector(s) comprises an a viral origin of replication, an expression cassette encoding at least one iPSC reprogramming factor and/or synthetic transcription factor, and a suicide gene; (b) culturing the first cell population to effect expression of the reprogramming factor(s) and/or synthetic transcription factor to produce a second cell population having traits consistent with embryonic stem cells; and (c) contacting the second cell population with a suicide gene substrate to produce a cell population that is essentially free of reprogramming vector(s). In embodiments, the suicide gene is selected from the group consisting of thymidine kinase and cytosine deaminase.

In embodiments of the methods that use a suicide gene to increase the efficiency of iPSC generation, after the episomal reprogramming vector(s) is introduced into the cell, the cells are cultured for a sufficiently long to allow the somatic cells to convert to iPSCs, i.e., cultured to effect expression of the reprogramming factors to produce a cell population having traits consistent with embryonic cells. In embodiments, the cells that have traits consistent with embryonic cells are subcultured. In embodiments, after subculture, the cells are further passaged before the suicide gene substrate is provided (GNC or 5-FC, for example). In additional embodiments, after subculture, the cells are not passaged before the suicide gene substrate is provided.

In embodiments, for use in the methods of the invention that use a suicide gene to increase efficiency of iPSC creation, the origin of replication is OriP. In further embodiments, the origin of replication is OriP and the expression cassette comprises a polynucleotide encoding EBNA-1 of EBV, or a derivative of EBNA-1 that has a deletion of residues 65 to 89 of EBNA-1, residues 90 to 328 of EBNA-1, or both.

In embodiments, the methods of the invention that use a suicide gene to increase efficiency of iPSC creation further include screening the cell population for the presence of the episomal reprogramming vector. Such screening can occur at any step in the method after the episomal reprogramming vector has been introduced into the somatic cells. In embodiments, the cells are screened following provision of the suicide gene substrate. Screening methods are known to those skilled in the art and include, but are not limited to qPCR vector detection assay. In embodiments, the cells in the cell population following suicide gene substrate provision that still contain an episomal reprogramming vector are not further cultured.

The methods of the invention further comprise producing induced human pluripotent stem cells (iPSCs) that are essentially free of episomal reprogramming vector(s), comprising: (a) introducing an episomal reprogramming vector(s) into a somatic cell to produce a first cell population, wherein the episomal reprogramming vector(s) comprises (i) an OriP replication origin, (ii) an expression cassette encoding an iPSC reprogramming factor and/or synthetic transcription factor, (iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1, and (iv) a thymidine kinase or cytosine deaminase suicide gene; (b) culturing the first cell population to effect expression of the reprogramming factor and/or synthetic transcription factor to produce a second cell population having traits consistent with embryonic stem cells; (c) contacting the second cell population with a suicide gene substrate to produce iPSCs that are essentially free of an episomal reprogramming vector. A description of EBNA-1 and derivatives is found in Levitskaya et al., Nature 375:685 (1995), Levitskaya et al., Proc. Natl. Acad. Sci. USA 94:12616-12621 (1997) and Yin, Science 301:1371-1374 (2003), each of which is incorporated herein by reference in its entirety.

In embodiments, the somatic cell for use in the methods of the invention that use a suicide gene to increase efficiency of iPSC creation include mononuclear cells, including human peripheral blood mononuclear cells, fibroblasts, keratinocytes, hematopoietic cells, mesenchymal cells, liver cells, stomach cells and/or β cells. In embodiments, the somatic cell for use in the methods of the invention that use a suicide gene to increase efficiency of iPSC creation are human peripheral blood mononuclear cells.

In embodiments, the reprogramming factors for use in the methods of the invention that use a suicide gene to increase efficiency of iPSC creation include Sox-2, Oct-4, Nanog, KLF4, cMYC, Lin-28, and/or p53DD. In additional embodiments, the iPSC reprogramming factors comprise Sox-2 and Oct-4. In embodiments, the iPSC reprogramming factors are Sox-2, Oct-4 and Nanog. In embodiments, the iPSC reprogramming factors are Sox-2, Oct-4, and one or more of KLF4, cMYC, Lin-28 and p53DD. In embodiments, the iPSC reprogramming factors are Sox-2, Oct-4, KLF4, cMYC, Lin-28 and p53DD.

In embodiments, when using the suicide gene approach alone to promote vector clearance, the appropriate substrate is added to the P0 plate, 4-5 days before colonies are picked. This will select for survival of vector-free colonies. A qPCR vector detection screening assay provides further verification as to which of the surviving iPSC colonies are promising for future expansion, banking and differentiation. As mentioned above, depending on vector clearance kinetics, it may be necessary to shift back suicide gene substrate addition to later passages.

Regulated Expression of EBNA-1

EBNA-1 is a viral protein which is involved in binding to a region termed OriP found in episomal reprogramming vectors and promoting tethering to chromosomal DNA during cell division. This results in increased plasmid maintenance following transfection. While this is beneficial in order to achieve high-level expression of reprogramming factors and/or synthetic transcription factors initially, it results in slow plasmid loss once reprogramming has occurred.

In embodiments, the invention provided herein regulates EBNA-1 either by repressing its transcription post-reprogramming (for example by using the tetracycline repressible (TetOff)) or by using an inducible system which is only activated until reprogramming has occurred (for example, tetracycline inducible (TetOn)). Alternative approaches also exist for controlling EBNA-1 expression (for example, an inducible antisense or interfering RNA to EBNA-1 could be expressed once reprogramming has occurred. Inducing agents such as doxycycline (Dox) (for Tet system) are available and compatible with current iPSC generation processes. These approaches reduce partitioning of plasmids during cell division and hence reduce the number of passages required to generate vector free cell lines.

In embodiments, the invention provides methods of producing induced human pluripotent stem cells (iPSCs) that are essentially free of reprogramming vector(s), comprising: (a) introducing a reprogramming vector(s) into a somatic cell to produce a first cell population, wherein the reprogramming vector comprises i) a viral origin of replication, ii) an expression cassette encoding iPSC reprogramming factor(s) and/or synthetic transcription factor(s), iii) a gene regulating extrachromosomal replication and partitioning of the reprogramming vector, and iv) a regulated promoter system; (b) culturing the first cell population to effect expression of the reprogramming factors and/or synthetic transcription factors to produce a second cell population having traits consistent with embryonic stem cells, wherein during culture of the first cell population the reprogramming vector is replicated; and (c) culturing the second cell population wherein the gene regulating extrachromosomal replication and partitioning of the reprogramming vector is regulated such that the reprogramming vector is lost during cell division, to produce iPSCs that are essentially free of the reprogramming vector.

In embodiments, the methods of the invention for regulating extrachromosomal replication and partitioning utilizes a tetracycline or tetracycline derivative activated system, e.g., a TetOn or TetOff system. In embodiments, when a TetOn system is used, tetracycline, or a tetracycline derivative such as doxycycline, is present while the transfected cell population is cultured to up regulate EBNA-1 expression. The cell population is then cultured in the absence of tetracycline, or a derivative, to reduce the expression of EBNA-1 and thus substantially reduce replication and partitioning of the episomal vector.

In embodiments, the methods of the invention for regulating extrachromosomal replication and partitioning utilize a TetOff system. In a TetOff system, tetracycline, or a tetracycline derivative such as doxycycline, is not present while the transfected cell population is cultured to up regulate EBNA-1 expression. The cell population is then cultured in the presence of tetracycline, or a derivative, to down regulate the expression of EBNA-1 and thus substantially reduce replication and partitioning of the episomal vector.

In embodiments, the methods of the invention regulating extrachromosomal replication and partitioning utilize an OriP origin of replication. In further embodiments, the origin of replication is OriP and the expression cassette comprises a polynucleotide encoding EBNA-1 of EBV, or a derivative of EBNA-1 that has a deletion of residues 65 to 89 of EBNA-1, residues 90 to 328 of EBNA-1, or both.

In embodiments, in the methods of the invention regulating extrachromosomal replication and partitioning, further include screening the cell population for the presence of the episomal reprogramming vector. Such screening can occur at any step in the method after the episomal reprogramming vector has been introduced into the somatic cells. In embodiments, the cells are screened following activation of the suicide gene. Screening methods are known to those skilled in the art and include, but are not limited to qPCR vector detection assay.

In embodiments of the methods regulating extrachromosomal replication and partitioning, after the episomal reprogramming vector is introduced into the cell, and the cells are cultured for a sufficiently long to allow the somatic cells to convert to iPSCs, i.e., cultured to effect expression of the reprogramming factors and/or synthetic transcription factors to produce a cell population having traits consistent with embryonic cells, the cells that have traits consistent with embryonic cells are subcultured. In embodiments, after subculture, the cells are further passaged before the cell population is cultured to produce iPSCs that are essentially free of the episomal reprogramming vector. In additional embodiments, after subculture, the cells are not passaged before culturing, wherein during culture, the episomal reprogramming vector is not replicated.

The invention further provides methods of producing induced human pluripotent stem cells (iPSCs) that are essentially free of episomal reprogramming vector(s), comprising: (a) introducing episomal reprogramming vector(s) into a somatic cell to produce a first cell population, wherein the episomal reprogramming vector comprises (i) an OriP replication origin, (ii) an expression cassette encoding an iPSC reprogramming factor and/or synthetic transcription factor, iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1, and (iv) a tetracycline or tetracycline derivative regulated promoter system (TetOn or TetOff); (b) culturing the first cell population to produce a second cell population having traits consistent with embryonic stem cells, wherein during culturing the episomal reprogramming vector is replicated; (c) culturing the second cell population to produce a third cell population, wherein during culturing the episomal reprogramming vector is not replicated; (d) selecting colonies from the third cell population to produce a fourth cell population; and (e) culturing the fourth cell population to produce iPSCs that are essentially free of the episomal reprogramming vector.

In embodiments, the methods of the invention regulating extrachromosomal replication and partitioning utilize a somatic cell that can be a human peripheral blood mononuclear cells, fibroblasts, keratinocytes, hematopoietic cells, mesenchymal cells, liver cells, stomach cells and (cells.

In embodiments, the reprogramming factors for use in the methods of the invention that regulate extrachromosomal replication and partitioning include Sox-2, Oct-4, Nanog, KLF4, cMYC, MYCl, Lin-28, and/or p53DD. In additional embodiments, the iPSC reprogramming factors comprise Sox-2 and Oct-4. In embodiments, the iPSC reprogramming factors are Sox-2, Oct-4 and Nanog. In embodiments, the iPSC reprogramming factors are Sox-2, Oct-4, and one or more of KLF4, cMYC, Lin-28 and pS3DD. In embodiments, the iPSC reprogramming factors are Sox-2, Oct-4, KLF4, cMYC, Lin-28 and p53DD.

In embodiments, when regulating EBNA-1 expression alone to promote vector clearance, EBNA-1 expression is reduced at a time-point corresponding to the P0 plate, before iPSC colonies are picked. As in the current iPSC generation process, colony picking is carried out, however, increased numbers of vector-free colonies are identified at earlier passage numbers.

Combination of Suicide Gene and Regulated Extrachromosomal Replication and Partioning

In embodiments, combining the methods provides additional advantages and allows beneficial modifications to the experimental approach. Due to the attributes of EBNA-1-based episomal vectors, providing a suicide gene substrate in the period of post-transfection (i.e. 20-30 days) results in extensive cell death of iPSC colonies present in the P0 plate. This undesirable effect is reduced by reducing EBNA-1 expression in order to promote vector clearance prior to adding the substrate for the suicide gene.

A further advantage of combining the methods is that when high reprogramming efficiency and high vector clearance are achieved, iPSC colonies can be picked as a pool, expediting expansion, banking and characterization.

The invention therefore further provides methods of producing induced human pluripotent stem cells (iPSCs) that are essentially free of reprogramming vector(s), comprising: (a) introducing a reprogramming vector(s) into a somatic cell to produce a first cell population, wherein the reprogramming vector comprises i) a viral origin of replication, ii) an expression cassette encoding iPSC reprogramming factor(s) and/or synthetic transcription factor(s), iii) a gene regulating extrachromosomal replication and partitioning of the reprogramming vector, iv) a regulated promoter system, and v) a suicide gene; (b) culturing the first cell population to effect expression of the reprogramming factor and/or synthetic transcription factor to produce a second cell population having traits consistent with embryonic stem cells, wherein during culture the reprogramming vector is replicated; (c) culturing the second cell population wherein the gene regulating extrachromosomal replication and partitioning is regulated such that the reprogramming vector is lost during cell division, to produce a third cell population comprising iPSCs that are substantially free of the reprogramming factor, and (d) contacting the third cell population with a suicide gene substrate to produce iPSCs that are essentially free of the reprogramming vector.

In the combination methods of the invention, in embodiments, the suicide gene is selected from the group consisting of thymidine kinase and cytosine deaminase.

In the combination methods of the invention, the origin of replication is OriP. In additional embodiments, the origin of replication is OriP and the expression cassette comprises a polynucleotide encoding EBNA-1 of EBV, or a derivative of EBNA-1 that has a deletion of residues 65 to 89 of EBNA-1, residues 90 to 328 of EBNA-1, or both.

In the combination methods of the invention, the gene regulating transcription of the expression cassette encoding iPSC reprogramming factors and/or synthetic transcription factors comprises a tetracycline or tetracycline derivative, e.g., doxycycline, activated system. In embodiments, doxycycline is present during culturing of the first cell population to effect expression of the reprogramming factors to produce a cell population having traits consistent with embryonic stem cells and doxycycline is absent during culturing to produce iPSCs that are substantially free of the episomal reprogramming vector, wherein during culture, the episomal reprogramming vector is not replicated. In embodiments, the doxycycline is absent during the first culturing step, and doxycycline is absent in the second step.

In the combination methods of the invention, additional embodiments further include screening the cell population for the presence of the episomal reprogramming vector. Such screening can occur at any step in the method after the episomal reprogramming vector has been introduced into the somatic cells. In embodiments, the cells are screened following culturing of the second cell population to produce a third cell population comprising iPSCs that are substantially free of an episomal reprogramming vector, wherein during culture of the second cell population, the episomal reprogramming vector is not replicated. In additional embodiments, the cells are screened following activation of the suicide gene. Screening methods are known to those skilled in the art and include, but are not limited to qPCR vector detection assay. In embodiments, after the second cell population is cultured to produce iPSCs that are essentially free of the episomal reprogramming vector, the cell population for the presence of the episomal reprogramming vector.

In embodiments of the combination methods of the invention, after the episomal reprogramming vector is introduced into the cell, and the cells are cultured for a sufficiently long to allow the somatic cells to convert to iPSCs, i.e., cultured to effect expression of the reprogramming factors and/or synthetic transcription factors to produce a cell population having traits consistent with embryonic cells, the cells that have traits consistent with embryonic cells are subcultured. In embodiments, after subculture, the cells are further passaged before the cell population is cultured to produce iPSCs that are essentially free of an episomal reprogramming vector. In additional embodiments, after subculture, the cells are not passaged before culturing, wherein during culture, the episomal reprogramming vector is not replicated. In additional embodiments of the combination methods of the invention, after the episomal reprogramming vector is introduced into the cell, the cells are cultured for a sufficiently long to allow the somatic cells to convert to iPSCs, i.e., cultured to effect expression of the reprogramming factors and/or synthetic transcription factors to produce a cell population having traits consistent with embryonic cells. In embodiments, the cells that have traits consistent with embryonic cells are subcultured. In embodiments, after subculture, the cells are further passaged before the suicide gene substrate is provided (adding GNC or 5-FC), for example. In additional embodiments, after subculture, the cells are not passaged before the suicide gene substrate is provided.

The invention further provides methods of producing induced human pluripotent stem cells (iPSCs) that are essentially free of episomal reprogramming vector(s), comprising: (a) introducing episomal reprogramming vector(s) into a somatic cell to produce a first cell population, wherein the episomal reprogramming vector comprises (i) an OriP replication origin, (ii) an expression cassette encoding an iPSC reprogramming factor and/or synthetic transcription factor, iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1, (iv) a tetracycline or tetracycline derivative regulated promoter system (TetOn or TetOff), and v) a thymidine kinase or cytosine deaminase suicide gene; (b) culturing the first cell population to effect expression of the reprogramming factors and/or synthetic transcription factors to produce a second cell population having traits consistent with embryonic stem cells, wherein during culturing the episomal reprogramming vector is replicated; (c) culturing the second cell population to produce a third cell population comprising iPSCs that are substantially free of the episomal reprogramming vector, wherein during culturing of the second cell population, the episomal reprogramming vector is not replicated; and (d) contacting the third cell population with a suicide gene substrate to produce iPSCs that are essentially free of the episomal reprogramming vector.

In some embodiments, the combination methods of the invention utilize a somatic cell that can be a human peripheral blood mononuclear cells, fibroblasts, keratinocytes, hematopoietic cells, mesenchymal cells, liver cells, stomach cells and β cells.

In some embodiments, the reprogramming factors for use in the combination methods of the invention include Sox-2, Oct-4, Nanog, KLF4, cMYC, Lin-28, and/or p53DD. In additional embodiments, the iPSC reprogramming factors comprise Sox-2 and Oct-4. In some embodiments, the iPSC reprogramming factors are Sox-2, Oct-4 and Nanog. In embodiments, the iPSC reprogramming factors are Sox-2, Oct-4, and one or more of KLF4, cMYC, Lin-28 and p53DD. In embodiments, the iPSC reprogramming factors are Sox-2, Oct-4, KLF4, cMYC, Lin-28 and p53DD.

Episomal Reprogramming Vectors

In embodiments, the invention provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a suicide gene.

In additional embodiments, the invention provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a thymidine kinase or cytosine deaminase suicide gene.

The invention further provides, in embodiments, an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a regulated promoter system.

In embodiments, the invention additionally provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and (d) a TetOn or TetOff system.

In embodiments, the invention additionally provides an episomal reprogramming vector comprising: (a) an OriP origin of replication; (b) an expression cassette encoding an iPSC reprogramming factor and/or synthetic transcription factor, (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; (d) a TetOn or TetOff system; and (e) a suicide gene.

In embodiments, the invention additionally provides an episomal reprogramming vector comprising: (a) an OriP origin of replication; (b) an expression cassette encoding an iPSC reprogramming factor and/or synthetic transcription factor; (c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; (d) a TetOn or TetOff system; and (e) a thymidine kinase or cytosine deaminase suicide gene.

In embodiments, the methods of the present invention utilize a single vector or multiple vectors to create iPSCs that are essentially free of the reprogramming vector. For example, in embodiments, the regulated EBNA-1 of EBV, or derivative of EBNA-1, as discussed above, is not on the same vector as the expression cassette reprogramming factors and/or the synthetic transcription factors. Likewise, in embodiments, the suicide gene and/or regulated promoter system are on separate vectors.

Reprogramming Factors

Reprogramming factors are necessary to produce iPSCs. The following factors or combination of factors can be used in the methods of the invention. In certain aspects, nucleic acids encoding Sox and Oct (particularly Oct3/4) will be included into the reprogramming vector. For example, one or more reprogramming vectors may comprise expression cassettes encoding Sox2, Oct4, Nanog and optionally Lin28, or expression cassettes encoding Sox2, Oct4, Klf4 and optionally c-Myc, or expression cassettes encoding Sox2, Oct4, and optionally Esrrb, or expression cassettes encoding Sox2, Oct4, Nanog, Lin28, Klf4, c-Myc, and optionally SV40 Large T antigen. Nucleic acids encoding these reprogramming factors may be comprised in the same expression cassette, different expression cassettes, the same reprogramming vector, or different reprogramming vectors.

Oct4 and certain members of the Sox gene family (Sox1, Sox2, Sox3, and Sox15) have been identified as crucial transcriptional regulators involved in the induction process whose absence makes induction impossible. Additional genes, however, including certain members of the Klf family (Klf1, Klf2, Klf4, and Klf5), the Myc family (c-Myc, L-Myc, and N-Myc), Nanog, and Lin28, have been identified to increase the induction efficiency.

Oct4 (Pou5fl) is one of the family of octamer (“Oct”) transcription factors, and plays a crucial role in maintaining pluripotency. The absence of Oct4 in cells, such as blastomeres and embryonic stem cells, leads to spontaneous trophoblast differentiation, and presence of Oct4 thus gives rise to the pluripotency and differentiation potential of embryonic stem cells. Various other genes in the “Oct” family, including Oct1 and Oct6, fail to elicit induction.

The Sox family of genes is associated with maintaining pluripotency similar to Oct4, although it is associated with multipotent and unipotent stem cells in contrast with Oct4, which is exclusively expressed in pluripotent stem cells. While Sox2 was the initial gene used for reprogramming induction, other genes in the Sox family have been found to work as well in the induction process. Sox1 yields iPS cells with a similar efficiency as Sox2, and genes Sox3, Sox15, and Sox18 also generate iPS cells, although with decreased efficiency.

Lin28 is an mRNA binding protein expressed in embryonic stem cells and embryonic carcinoma cells associated with differentiation and proliferation.

The reprogramming factors used in the methods and vectors of the invention can be naturally or non-naturally occurring. Non-naturally occurring reprogramming factors are referred to herein as synthetic transcription factors. Synthetic transcription factors can also be introduced into somatic cells to reprogram the cells into an embryonic stem cell-like state. Synthetic transcription factors can enhance reprogramming efficiency and accelerate kinetics. Additional synthetic transcription factors are known to one of skill in the art.

The reprogramming proteins used in the present invention can be substituted by protein homologs with about the same reprogramming functions. Nucleic acids encoding those homologs could also be used for reprogramming. Conservative amino acid substitutions are preferred—that is, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide.

Suicide Genes

Suicide genes have been tested in cancer therapy. A major limitation of conventional chemotherapies used in cancer treatments today are low therapeutic indices and side effects that result from drug effects on normal tissues. One of the most innovative approaches to developing therapies with increased tumor selectivity is gene therapy.

The fundamental concept underlying suicide gene therapy is as follows: a gene is selectively introduced into the tumor environment which encodes for an enzyme that metabolizes a systemically available pro-drug to an active anti-neoplastic agent locally. The first example of suicide gene approach for therapy of cancer was the introduction of the herpes simplex virus thymidine kinase gene into neoplastic BALB/c murine K3T3 sarcoma cell lines. Treatment with ganciclovir, which is converted by thymidine kinase into compounds that become toxic after triphosphorylation by cellular kinases, resulted in destruction of the tumor cells in vitro. Administration of ganciclovir to BALB/c mice bearing K3T3 sarcoma tumors produced by the cell lines resulted in destruction of the tumors in vivo. The central rationale for suicide gene therapy is to artificially generate exploitable biochemical differences between healthy host tissues and cancer cells.

In the captioned invention, suicide genes are exploited to eliminate iPSCs that still contain the episomal reprogramming vector. When the suicide gene systems are activated, only those cells containing the episomal vector are killed.

Extra-Chromosomal Vectors for Generating Vector-Free Induced Pluripotent Stem Cells

As described above, generation of pluripotent stem cells from human somatic cells has been achieved using retroviruses or lentiviral vectors for ectopic expression of reprogramming genes. Recombinant retroviruses such as the Moloney murine leukemia virus have the ability to integrate into the host genome in a stable fashion. They contain a reverse transcriptase which allows integration into the host genome. Lentiviruses are a subclass of Retroviruses. They are widely adapted as vectors thanks to their ability to integrate into the genome of non-dividing as well as dividing cells. These viral vectors also have been widely used in a broader context: differentiation programming of cells, including reprogramming, differentiation, and transdifferentiation. The viral genome in the form of RNA is reverse-transcribed when the virus enters the cell to produce DNA, which is then inserted into the genome at a random position by the viral integrase enzyme. As discussed above, integration of the viral nucleotides into the host genome is not preferred for cell therapy based therapeutic applications.

Therefore, in certain embodiments, the present invention provides methods to generate induced pluripotent stem cells and other desired cell types essentially free of exogenous genetic elements, such as from retroviral or lentiviral vector used in the previous methods. These methods make use of extra-chromosomally replicating vectors, or vectors capable of replicating episomally.

Epstein-Barr Virus

The Epstein-Barr Virus (EBV), also called Human herpesvirus 4 (HHV-4), is a virus of the herpes family (which includes Herpes simplex virus and Cytomegalovirus). EBV maintains its genome extra-chromosomally and works in collaboration with host cell machinery for efficient replication and maintenance relying solely on two essential features for its replication and its retention within cells during cell division. One element, OriP, exists in cis and serves as the origin of replication. The other factor, EBNA1, functions in trans by binding to sequences within OriP to promote replication and maintenance of the plasmid DNA.

OriP

OriP is a region of the Epstein-Barr virus chromosome that supports the replication and stable maintenance of plasmids in human cells. It is the site at or near which DNA replication initiates and is composed of two cis-acting sequences approximately 1 kilobase pair apart known as the family of repeats (FR) and the dyad symmetry (DS).

FR is composed of 21 imperfect copies of a 30 bp repeat and contains 20 high affinity EBNA1-binding sites. When FR is bound by EBNA1, it both serves as a transcriptional enhancer of promoters in cis up to 10 kb away, and contributes to the nuclear retention and faithful maintenance of FR containing plasmids. The efficient partitioning of OriP plasmids is also likely attributable to FR. While the virus has evolved to maintain 20 EBNA1-binding sites in FR, efficient plasmid maintenance requires only seven of these sites, and can be reconstituted by a polymer of three copies of DS, having a total of 12 EBNA1-binding sites.

The dyad symmetry element (DS) is sufficient for initiation of DNA synthesis in the presence of EBNA1, and initiation occurs either at or near DS. Termination of viral DNA synthesis is thought to occur at FR, because when FR is bound by EBNA1 it functions as a replication fork barrier as observed by 2D gel electrophoresis. Initiation of DNA synthesis from DS is licensed to once-per-cell-cycle, and is regulated by the components of the cellular replication system. DS contains four EBNA1-binding sites, albeit with lower affinity than those found in FR. The topology of DS is such that the four binding sites are arranged as two pairs of sites, with 21 bp center-to-center spacing between each pair and 33 bp center-to-center spacing between the two non-paired internal binding sites.

The functional roles of the elements within DS have been confirmed by studies of another region of EBV's genome, termed Rep*, which was identified as an element that can substitute for DS inefficiently. Polymerizing Rep* eight times yielded an element as efficient as DS in its support of replication. Biochemical dissection of Rep* identified a pair of EBNA 1-binding sites with a 21 bp center-to-center spacing critical for its replicative function (ibid). The minimal replicator of Rep* was found to be the pair of EBNA1-binding sites, as replicative function was retained even after all flanking sequences in the polymer were replaced with sequences derived from lambda phage. Comparisons of DS and Rep* have revealed a common mechanism: these replicators support the initiation of DNA synthesis by recruiting the cellular replicative machinery via a pair of appropriately spaced sites, bent and bound by EBNA1.

Additional extra-chromosomal, plasmids that replicate in mammalian cells exist that are unrelated to EBV and appear similar to the zone of initiation within the Raji strain of EBV. For example, plasmids that contain “nuclear scaffold/matrix attachment regions” (S/MARs) and a robust transcriptional unit are in the art. Their S/MAR is derived from the human interferon-beta gene, is A/T rich, and operationally defined by its association with the nuclear matrix and its preferential unwinding at low ionic strength or when embedded in supercoiled DNA. These plasmids replicate semiconservatively, bind ORC proteins, and support the initiation of DNA synthesis effectively randomly throughout their DNA. They are efficiently maintained in proliferating hamster and human cells without drug selection and when introduced into swine embryos can support expression of GFP in most tissues of fetal animals.

EBNA1

Epstein Barr nuclear antigen 1 (EBNA1) is a DNA-binding protein that binds to FR and DS of OriP or Rep* to facilitate replication and faithful partitioning of the EBV plasmid to daughter cells independent of; but in concert with, cell chromosomes during each cell division.

The 641 amino acids (AA) of EBNA1 have been categorized into domains associated with its varied functions by mutational and deletional analyses. Two regions, between AA40-89 and AA329-378 are capable of linking two DNA elements in cis or in trans when bound by EBNA1, and have thus been termed Linking Region 1 and 2 (LR1, LR2). Fusing these domains of EBNA1 to GFP homes the GFP to mitotic chromosomes. LR1 and LR2 are functionally redundant for replication; a deletion of either one yields a derivative of EBNA1 capable of supporting DNA replication. LR1 and LR2 are rich in arginine and glycine residues, and resemble the AT-hook motifs that bind A/T rich DNA. An in vitro analysis of LR1 and LR2 of EBNA1 has demonstrated their ability to bind to A/T rich DNA. When LR1, containing one such AT-hook, was fused to the DNA-binding and dimerization domain of EBNA1, it was found to be sufficient for DNA replication of OriP plasmids, albeit less efficiently than the wild-type EBNA1.

LR2 is not required for EBNA1's support of OriP replication. Additionally, the N-terminal half of EBNA1 can be replaced with cellular proteins containing AT-hook motifs, such as HMGA1a, and still retain replicative function. These findings indicate that it likely is the AT-hook activities of LR1 and LR2 are required for the maintenance of OriP in human cells.

A third of EBNA1's residues (AA91-328) consist of glycine-glycine-alanine (GGA) repeats, implicated in EBNA1's ability to evade the host immune response by inhibiting proteosomal degradation and presentation. These repeats have also been found to inhibit translation of EBNA1 in vitro and in vivo. However, the deletion of much of this domain has no apparent effect on functions of EBNA1 in cell culture.

A nuclear localization signal (NLS) is encoded by AA379-386, which also associates with the cellular nuclear importation machinery.

Lastly, the C-terminus (AA458-607) encodes the overlapping DNA-binding and dimerization domains of EBNA1. The structure of these domains bound to DNA has been solved by X-ray crystallography, and was found to be similar to the DNA-binding domain of the E2 protein of papillomaviruses.

In embodiments of the invention, a reprogramming vector will contain both OriP and an abbreviated sequence encoding a version of EBNA1 competent to support plasmid replication and its proper maintenance during cell division. The highly repetitive sequence within the amino-terminal one-third of wild-type EBNA1 and removal of a 25 amino-acid region that has demonstrated toxicity in various cells are dispensable for EBNA1's trans-acting function associated with OriP. Therefore, an exemplary derivative, the abbreviated form of EBNA1, known as deltaUR1, could be used with OriP within this plasmid-based system. More examples of EBNA1 derivatives that can activate transcription from an extra-chromosomal template are available in the art, for example, Kirchmaier and Sugden, J. Virol. 71(3):1776-1775 (1997), and Kennedy and Sugden, Mol. Cell. Biol. 23(19):6901-6908 (2003), both incorporated herein by reference.)

A derivative of EBNA-1 used in the invention is a polypeptide which, relative to a corresponding wild-type polypeptide, has a modified amino acid sequence. The modifications include the deletion, insertion or substitution of at least one amino acid residue in a region corresponding to the unique region (residues about 65 to about 89) of LR1 (residues about 40 to about 89) in EBNA-1, and may include a deletion, insertion and/or substitution of one or more amino acid residues in regions corresponding to other residues of EBNA-1, e.g., about residue 1 to about residue 40, residues about 90 to about 328 (“Gly-Gly-Ala” repeat region), residues about 329 to about 377 (LR2), residues about 379 to about 386 (NLS), residues about 451 to about 608 (DNA binding and dimerization), or residues about 609 to about 641, so long as the resulting derivative has the desired properties, e.g., dimerizes and binds DNA containing an ori corresponding to OriP, localizes to the nucleus, is not cytotoxic, and activates transcription from an extrachromosomal but does not substantially active transcription from an integrated template. Substitutions include substitutions which utilize the D rather than L form, as well as other well known amino acid analogs, e.g., unnatural amino acids such as alpha-disubstituted amino acids, N-alkyl amino acids, lactic acid, and the like. These analogs include phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, alpha-methyl-alanine, para-benzoyl-phenylalanine, phenylglycine, propargylglycine, sarcosine, epsilon-N,N,N-trimethyllysine, epsilon-N-acetyllysine, N-acetylserine, N-formylmethionine, 3-methylhistidine, 5-hydroxylysine, omega-N-methylarginine, and other similar amino acids and imino acids and tert-butylglycine.

Conservative amino acid substitutions are preferred—that is, for example, aspartic-glutamic as polar acidic amino acids; lysine/arginine/histidine as polar basic amino acids; leucine/isoleucine/methionine/valine/alanine/glycine/proline as non-polar or hydrophobic amino acids; serine/threonine as polar or uncharged hydrophilic amino acids. Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting polypeptide. Whether an amino acid change results in a functional polypeptide can readily be determined by assaying the specific activity of the polypeptide.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic; trp, tyr, phe.

The invention also envisions polypeptides with non-conservative substitutions. Non-conservative substitutions entail exchanging a member of one of the classes described above for another.

Acid addition salts of the polypeptide or of amino residues of the polypeptide may be prepared by contacting the polypeptide or amine with one or more equivalents of the desired inorganic or organic acid, such as, for example, hydrochloric acid. Esters of carboxyl groups of the polypeptides may also be prepared by any of the usual methods known in the art.

Vector Construction and Delivery

In certain embodiments, reprogramming or differentiation programming vectors are constructed to comprise additional elements in addition to nucleic acid sequences encoding reprogramming factors, differentiation programming factors and/or synthetic transcription factors as described above to express these reprogramming factors in cells. In embodiments, the components of these vectors and delivery methods are disclosed below.

Vector

The use of plasmid- or liposome-based extra-chromosomal vectors, e.g., OriP-based vectors, and/or vectors encoding a derivative of EBNA-1 permit large fragments of DNA to be introduced to a cell and maintained extra-chromosomally

Other extra-chromosomal vectors include other lymphotrophic herpes virus-based vectors. Lymphotrophic herpes virus is a herpes virus that replicates in a lymphoblast (e.g., a human B lymphoblast) and becomes a plasmid for a part of its natural life-cycle. Exemplary lymphotrophic herpes viruses include, but are not limited to EBV, Kaposi's sarcoma herpes virus (KSHV); Herpes virus saimiri (HS) and Marek's disease virus (MDV). Also other sources of episome-base vectors are provided, such as yeast ARS, adenovirus, SV40, or BPV.

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Regulatory Elements

Eukaryotic expression cassettes included in the vectors preferably contain (in a 5′-to-3′ direction) a eukaryotic transcriptional promoter operably linked to a protein-coding sequence, splice signals including intervening sequences, and a transcriptional termination/polyadenylation sequence.

Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

Promoters suitable for use in EBNA-1-encoding vector of the invention are those that direct the expression of the expression cassettes encoding the EBNA-1 protein to result in sufficient steady-state levels of EBNA-1 protein to stably maintain EBV OriP-containing vectors. Promoters are also used for efficient expression of expression cassettes encoding reprogramming factors and/or synthetic transcription factors.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the beta-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR, in connection with the compositions disclosed herein. Furthermore, in embodiments, the control sequences that direct transcription and/or expression of sequences within non-nuclear organelles such as mitochondria, chloroplasts, and the like, can be employed as well.

It is be important to employ a promoter and/or enhancer that effectively directs the expression of the DNA segment in the organelle, cell type, tissue, organ, or organism chosen for expression. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Use of a T3, T7 or SP6 cytoplasmic expression system is one embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, S. Y., Nuc. Acid Res. 17: 601-615, 1989, Quitsche et al., J. Biol. Chem. 264: 9539-9545, 1989), GADPH promoter (Alexander et al., Proc. Nat. Acad. Sci. USA 85: 5092-5096, 1988, Ercolani et al., J. Biol. Chem. 263: 15335-15341, 1988), metallothionein promoter (Karin et al. Cell 36: 371-379, 1989; Richards et al., Cell 37: 263-272, 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example is the phosphoglycerate kinase (PGK) promoter.

Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

Multiple Cloning Sites

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available.

Splicing Sites

In embodiments, the vectors used in the methods of the invention comprise a splicing site. Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression.

Termination Signals

In embodiments, the vectors or constructs of the present invention comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is provided. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators provided for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

Selection and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is calorimetric analysis, are also provided. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art. One feature of the present invention includes using selection and screenable markers to select vector-free cells after the differentiation programming factors have effected a desired altered differentiation status in those cells.

Vector Delivery

Introduction of a reprogramming or differentiation programming vector into somatic cells with the current invention may use any suitable methods for nucleic acid delivery for transformation of a cell, as described herein or as would be known to one of ordinary skill in the art. Such methods include, but are not limited to, direct delivery of DNA such as by ex vivo transfection, including microinjection; by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by PEG-mediated transformation of protoplasts; by desiccation/inhibition-mediated DNA uptake, and any combination of such methods. Additional methods for delivering the vectors of the present invention include “cell squeezing,” which involves rapid mechanical deformation of cells to deliver macromolecules and nanomaterials into cells. See, e.g., Sharei, “Cell Squeezing as a Robust, Microfluidic Intracellular Delivery Platform,” J. Vis. Exp. 81:1-7 (2013), incorporated herein by reference in its entirety.

Liposome-Mediated Transfection

In a certain embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also provided is an nucleic acid complexed with Lipofectamine (Gibco BRL) or Superfect (Qiagen). The amount of liposomes used may vary upon the nature of the liposome as well as the, cell used, for example, about 5 to about 20 μg vector DNA per 1 to 10 million of cells may be used.

Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 microgram vector DNA per 1 to 10 million of cells may be provided.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

Calcium Phosphate

In other embodiments of the present invention, a nucleic acid is introduced to the cells using calcium phosphate precipitation. Human KB cells have been transfected with adenovirus 5 DNA using this technique. Also in this manner, mouse L(A9), mouse C127, CHO, CV-1, BHK, NIH3T3 and HeLa cells were transfected with a neomycin marker gene, and rat hepatocytes were transfected with a variety of marker genes.

DEAE-Dextran

In another embodiment, a nucleic acid is delivered into a cell using DEAE-dextran followed by polyethylene glycol. In this manner, reporter plasmids were introduced into mouse myeloma and erythroleukemia cells.

Sonication Loading

Additional embodiments of the present invention include the introduction of a nucleic acid by direct sonic loading. LTK-fibroblasts have been transfected with the thymidine kinase gene by sonication loading.

Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EOF) is used in the receptor-mediated delivery of a nucleic acid to cells that exhibit upregulation of the EOF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is provided that the tissue-specific transforming constructs of the present invention can be specifically delivered into a target cell in a similar manner.

Microprojectile Bombardment

Microprojectile bombardment techniques can be used to introduce a nucleic acid into at least one, organelle, cell, tissue or organism. This method depends on the ability to accelerate DNA-coated microprojectiles to a high velocity allowing them to pierce cell membranes and enter cells without killing them. There are a wide variety of microprojectile bombardment techniques known in the art, many of which are applicable to the invention.

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force. The microprojectiles used have consisted of biologically inert substances such as tungsten or gold particles or beads. Exemplary particles include those comprised of tungsten, platinum, and preferably, gold. It is provided that in some instances DNA precipitation onto metal particles would not be necessary for DNA delivery to a recipient cell using microprojectile bombardment. However, it is provided that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

Selection of iPS Cells

In certain aspects of the invention, after a reprogramming vector is introduced into somatic cells, cells will be cultured for expansion (optionally selected for the presence of vector elements like positive selection or screenable marker to concentrate transfected cells) and reprogramming vectors will express reprogramming factors in these cells and replicate and partition along with cell division. These expressed reprogramming factors will reprogram somatic cell genome to establish a self-sustaining pluripotent state, and in the meantime or after removal of positive selection of the presence of vectors, exogenous genetic elements will be lost gradually. These induced pluripotent stem cells could be selected from progeny derived from these somatic cells based on embryonic stem cell characteristics because they are expected to share similar characteristics with pluripotent embryonic stem cells. An additional negative selection step could be also employed to accelerate or help selection of iPSC cells essentially free of exogenous genetic elements by testing the absence of reprogramming vector DNA or using selection markers.

Selection for Embryonic Stem Cell Characteristics

iPSCs are similar to naturally-isolated pluripotent stem cells (such as mouse and human embryonic stem cells, mESCs and hESCs, respectively) in the following respects, thus confirming the identity, authenticity, and pluripotency of iPSCs to naturally-isolated pluripotent stem cells. Thus, induced pluripotent stem cells generated from the methods disclosed in this invention could be selected based on one or more of following embryonic stem cell characteristics.

Cellular Biological Properties

Morphology: iPSCs are morphologically similar to ESCs. Each cell may have round shape, large nucleolus and scant cytoplasm. Colonies of iPSCs could be also similar to that of ESCs. Human iPSCs form sharp-edged, flat, tightly-packed colonies similar to hESCs and mouse iPSCs form the colonies similar to mESCs, less flatand more aggregated colonies than that of hESCs.

Growth properties: Doubling time and mitotic activity are cornerstones of ESCs, as stem cells must self-renew as part of their definition. iPSCs could be mitotically active, actively self-renewing, proliferating, and dividing at a rate equal to ESCs.

Stem Cell Extracellular Markers: iPSCs may express cell surface antigenic markers expressed on ESCs. Human iPSCs expressed the markers specific to hESC, including, but not limited to, SSEA-3, SSEA-4, TRA-1-60, TRA-1-81, TRA-2-49/6E. Mouse iPSCs expressed SSEA-1 but not SSEA-3 nor SSEA-4, similarly to mESCs.

Stem Cell Genes: iPSCs may express genes expressed in undifferentiated ESCs, including Oct-3/4, Sox-2, Nanog, GDF3, REX1, FGF4, ESG1, DPPA2, DPPA4, and hTERT.

Telomerase Activity: Telomerases are necessary to sustain cell division unrestricted by the Hayflick limit of .about.50 cell divisions. hESCs express high telomerase activity to sustain self-renewal and proliferation, and iPSCs also demonstrate high telomerase activity and express hTERT (human telomerase reverse transcriptase), a necessary component in the telomerase protein complex.

Pluripotency: iPSCs will be capable of differentiation in a fashion similar to ESCs into cells of the three germ layers.

Neural Differentiation: iPSCs could be differentiated into neurons, expressing betaIII-tubulin, tyrosine hydroxylase, AADC, DAT, ChAT, LMX1B, and MAP2. The presence of catecholamine-associated enzymes may indicate that iPSCs, like hESCs, may be differentiable into dopaminergic neurons. Stem cell-associated genes will be downregulated after differentiation.

Cardiac Differentiation: iPSCs could be differentiated into cardiomyocytes that spontaneously began beating. Cardiomyocytes expressed TnTc, MEF2C, MYL2A, MYHCbeta, and NKX2.5. Stem cell-associated genes will be downregulated after differentiation.

Teratoma Formation: iPSCs injected into immunodeficient mice may spontaneously formed teratomas after certain time, such as nine weeks. Teratomas are tumors of multiple lineages containing tissue derived from the three germ layers endoderm, mesoderm and ectoderm; this is unlike other tumors, which typically are of only one cell type. Teratoma formation is a landmark test for pluripotency.

Embryoid Body: hESCs in culture spontaneously form ball-like embryo-like structures termed “embryoid bodies,” which consist of a core of mitotically active and differentiating hESCs and a periphery of fully differentiated cells from all three germ layers. iPSCs may also form embryoid bodies and have peripheral differentiated cells.

Blastocyst Injection: hESCs naturally reside within the inner cell mass (embryoblast) of blastocysts, and in the embryoblast, differentiate into the embryo while the blastocyst's shell (trophoblast) differentiates into extraembryonic tissues. The hollow trophoblast is unable to form a living embryo, and thus it is necessary for the embryonic stem cells within the embryoblast to differentiate and form the embryo. iPSCs injected by micropipette into a trophoblast to generate a blastocyst transferred to recipient females, may result in chimeric living mouse pups: mice with iPSC derivatives incorporated all across their bodies with 10%-90 and chimerism.

Epigenetic Reprogramming

Promoter Demethylation: Methylation is the transfer of a methyl group to a DNA base, typically the transfer of a methyl group to a cytosine molecule in a CpG site (adjacent cytosine/guanine sequence). Widespread methylation of a gene interferes with expression by preventing the activity of expression proteins or recruiting enzymes that interfere with expression. Thus, methylation of a gene effectively silences it by preventing transcription. Promoters of pluripotency-associated genes, including Oct-3/4, Rex1, and Nanog, may be demethylated in iPSCs, showing their promoter activity and the active promotion and expression of pluripotency-associated genes in iPSCs.

Histone Demethylation: Histones are compacting proteins that are structurally localized to DNA sequences that can effect their activity through various chromatin-related modifications. H3 histones associated with Oct-3/4, Sox-2, and Nanog may be demethylated to activate the expression of Oct-3/4, Sox-2, and Nanog.

Selection for Residue Free Feature

A reprogramming vector such as OriP-based plasmid in this invention will replicate extra-chromosomally and lose it presence in host cells after generations. However, an additional selection step for progeny cells essentially free of exogenous vector elements may facilitate this process. For example, a sample of progeny cell may be extracted to test the presence or loss of exogenous vector elements as known in the art.

An alternative or complementary approach is to test the absence of exogenous genetic elements in progeny cells, using conventional methods, such as RT-PCR, PCR, FISH (Fluorescent in situ hybridization), gene array, or hybridization (e.g., Southern blot).

Culturing of iPS Cells

After somatic cells are introduced with a reprogramming vector using the disclosed methods, these cells may be cultured in a medium sufficient to maintain the pluripotency. Culturing of induced pluripotent stem (iPS) cells generated in this invention can use various medium and techniques developed to culture primate pluripotent stem cells, more specially, embryonic stem cells.

For example, like human embryonic stem (hES) cells, iPS cells can be maintained in 80% DMEM (Gibco #10829-018 or #11965-092), 20% defined fetal bovine serum (FBS) not heat inactivated, 1% non-essential amino acids, 1 mM L-glutamine, and 0.1 mM beta-mercaptoethanol. Alternatively, ES cells can be maintained in serum-free medium, made with 80% Knock-Out DMEM (Gibco #10829-018), 20% serum replacement (Gibco #10828-028), 1% non-essential amino acids, 1 mM L-glutamine, and 0.1 mM beta-mercaptoethanol. Just before use, human bFGF is added to a final concentration of .about 4 ng/mL (WO 99/20741).

IPS cells, like ES cells, have characteristic antigens that can be identified by immunohistochemistry or flow cytometry, using antibodies for SSEA-1, SSEA-3 and SSEA-4 (Developmental Studies Hybridoma Bank, National Institute of Child Health and Human Development, Bethesda Md.), and TRA-1-60 and TRA-1-81 (Andrews at al., in Robertson E, ed. Teratocarcinomas and Embryonic Stem Cells. IRL Press, 207-246, 1987). Pluripotency of embryonic stem cells can be confirmed by injecting approximately 0.5-10 10 6 cells into the rear leg muscles of 8-12 week old male SCID mice. Teratomas develop that demonstrate at least one cell type of each of the three germ layers.

### EXAMPLES

The invention is now described with reference to the following examples. These examples are illustrative only and the invention should in no way be construed as being limited to these examples but rather should be construed to encompass any and all variations which become evident as a result of the teachings provided herein.

### Example 1

### Materials and Methods

Protocol: Feeder-Independent Reprogramming of Human PBMCs with Episomal Plasmids and Reprogramming Enhancer A Using the 4D Nucleofector

Materials:
1. hPBMCs (Lonza Cat. CC-2702, (50×10⁶ cells/vial)
2. Lonza L7 hPSC Culture Medium™ and Supplement Kit
3. Lonza L13 hPSC Passaging Solution™
4. Lonza L7 hPSC Matrix™
5. Lonza 4D Nucleofector™
6. Lonza P3 Primary Cell 4D-Nucleofector™ Kit
7. Lonza Episomal Reprogramming Kit™
   a. Episomal Reprogramming Plasmid Mix™
   b. Episomal Enhancer A™
8. 6- and 12-well Tissue Culture Treated Plates
9. PBMC Basal Medium: HPGM™; Poietics™ hematopoietic progenitor growth medium Without antibiotics
10. PBMC Medium Supplements (see table)
11. Centrifuge Tubes
12. 1×PBS
13. Dry Ice
14. Parafilm™
15. Low Oxygen Humidified Cell Culture Incubator (3% O₂; 5% CO₂)
16. Humidified Cell Culture Incubator (20.9% O₂; 5% CO₂)

During priming steps (Day 0-6) a significant decrease in cell number is observed. This protocol is optimized for 10-50×10′ starting cell number. If priming is less than 10×10⁶ cells, consider priming the cells for an additional 2 days, nucleofecting on day 8.

Supplemented HPGM is good up to 10 days, when kept at 4° C. All centrifugation should be performed at room temperature.

### Procedure

Plan and estimate quantity of PBMC Basal Medium and PBMC Medium with supplements that will be needed each day. Bring quantity needed to the appropriate temperature.

Day 0: Remove vial of hPBMCs from liquid nitrogen tank. Promptly begin to thaw vial in 37° C. water bath without submerging cap. When there is a very small amount of ice visible, remove vial from water bath. Cleanse outside of vial with 70% ethanol.

Transfer contents of vial to a 50 ml centrifuge tube.

Slowly add 49 ml of room temperature Basal PBMC Medium, dropwise, to cells. Do not add a large volume of medium to the cells at one time. This may result in osmotic shock.

Collect cells at 200×g for 15 minutes.

Remove media from the tube, without disturbing the cell pellet.

Gently suspend the cell pellet in 10 ml PBMC Medium, containing all supplements. Count cells.

Seed cells into 6-well tissue culture treated plate at 2-4×10⁶ cells/ml.

Place plate in a humidified 37° C. incubator under normoxic conditions (20.9% O₂; 5% CO₂).

Day 3: Transfer the cells to a 15 ml centrifuge tube. Rinse well with 1 ml Basal PBMC Medium. Collect the cells at 200×g for 5 minutes. Remove media from the tube, without disturbing the cell pellet.

Gently suspend the cell pellet in 10 ml PBMC Medium, containing all supplements. Count cells.

Seed cells into 6-well plate at 0.5-1×10⁶ cells/ml.

Place plate in a humidified 37° C. incubator under normoxic conditions (20.9% O₂; 5% CO₂).

Day 5: Prepare 6-well plate with L7 hPSC Matrix™ for Day 6 according to the instruction insert.

Day 6: Remove matrix solution from 6-well plate. Add 2 ml PBMC Medium, containing all supplements, to each well needed. Add 6 μl Episomal Enhancer A™ to each well.

Pre-equilibrate in a hypoxic humidified incubator at 37° C. (3% O₂; 5% CO₂) for one hour.

Remove cells from incubator. Transfer to 15 ml tube. Rinse well with 1 ml Basal PBMC Medium. Count cells.

Transfer 1×10⁶ cells to at least two 15 ml tubes.

One tube will not be nucleofected and will be prepared as a genomic DNA control. This control can later be used to confirm the identity of the iPSC line (STR analysis).

Transfer tube to centrifuge and collect cells at 200×g for 5 minutes. Set control tube aside until the nucleofection reactions are completed.

For each nucleofection: Pipet 100 μl P3 Nucleofector™ Solution into one tube containing 3 ug of Episomal Reprogramming Plasmid Mix™.

Remove supernatant from cells that have been collected in centrifuge tubes.

Suspend each tube of cells in the prepared nucleofection reagents (Step 20).

Cell exposure to P3 Nucleofector™ Solution should be minimized. Prepare and process only one tube at a time through the 4D Nucleofector™.

Transfer cells to Nucleocuvette™ and place in 4D Nucleofector™. Avoid creating bubbles Nucleofect cells using program EO-115.

Using transfer pipet, supplied with Nucleofection™ Kit, add approximately 500 μl pre-warmed PBMC Medium (containing all supplements) to the cuvette and transfer cells directly into one well of the equilibrated 6-well plate.

Place cells in a hypoxic humidified incubator at 37° C. (3% O₂; 5% CO₂) for two days.

Finish processing genomic DNA control sample: Remove medium from tube containing cells set aside in step 18. Using a 1 ml serological pipet, Suspend cells in 1×PBS and transfer to a 1.5 ml centrifuge tube. Centrifuge cells at 300×g for 5 min. Carefully remove supernatant. Flash-freeze cell pellet on dry ice and store sample at −80° C.

Day 8: Add 2 ml L7 hPSC Culture Medium™, containing supplement to each well with nucleofected cells.

Place cells in a hypoxic humidified incubator at 37° C. (3% O₂; 5% CO₂) for two days.

Day 10: Replace medium with 2 ml L7 hPSC Culture Medium™, containing supplement

Place cells in a hypoxic humidified incubator at 37° C. (3% O₂; 5% CO₂) for two days.

Continue with every-other-day media changes starting on Day 14: Replace medium with 2 ml L7 hPSC Culture Medium™, containing supplement. Repeat medium change every other day until colonies are large enough to subculture.

PBMC Medium Supplements (to add to HPGM for
priming and recovery after nucleofection)

| | | Stock | Final | |
|---|---|---|---|---|
| Component | Vendor | Conc. | Conc. | |
| 1-Thioglycerol | Sigma | | 200 | μM |
| | #M6145 | | | |
| Holo- | R&D | 20 mg/ml | 100 | μg/ml |
| transferrin | Systems | | | |
| | #2914-HT | | | |
| Dexamethasone | Sigma | 10 mM | 1 | μM |
| | #D1756 | (10,000X) | | |
| SCF | PeproTech | 100 ug/ml | 100 | ng/ml |
| | #300-07 | (2,000X) | | |
| EPO | R&D | 2 U/μl | 2 | U/ml |
| | Systems | (1,000X) | | |
| | #287-TC- | | | |
| | 500 | | | |
| IL-3 | PeproTech | 10 μg/ml | 10 | ng/ml |
| | #200-03 | | | |
| IGF-1 | Peprotech | 40 ng/μl | 40 | ng/ml |
| | #100-11 | | | |

Subculturing iPSC colonies: Prepare 12-well plate(s) with L7 hPSC Matrix™.

Manually passage (P1) the initial colonies present into separate wells using L7 hPSC Culture Medium™, containing supplement.

Place plate in a humidified 37° C. incubator under normoxic conditions (20.9% O₂; 5% CO₂).

Human iPSC cultures should be cultivated in a humidified 37° C. incubator under normoxic conditions (20.9% O₂; 5% CO₂) once colonies have been manually passaged to new plates (step 36).

For P3 and later passages, use L7 hPSC Passaging Solution™, according to the instruction insert, to subculture colonies during expansion.

Step 1, Experimental goal 1: Determine the Kinetics of Vector Clearance in iPSCs by Regulating EBNA-1 Expression

For this experiment the EBNA-1 sequence will be cloned downstream of the TRE promoter from a functional TetOn vector to create the TetOn-EBNA-1 vector. In this system EBNA-1 expression will be activated in the presence of doxycycline (Dox) (TetOn system). Using the same cloning strategy, TetOn-eGFP vector will be created to use as a control vector for Tet regulation. To test the effect of regulated vs. constitutive EBNA-1 expression, a vector containing the CAG promoter driving EBNA-1 expression will be generated. Both the TetOn-EBNA-1 vector and CAG-EBNA-1 vectors will be tested with and without the OriP region. Finally, the ‘Test’ vector contains a constitutive eGFP expression cassette (SV40 promoter) and contains the OriP region. The ‘Test’ vector is the mimic of the standard vectors containing the reprogramming factors. See Table 1 for list and description of vectors that will be used for exploring the effect of EBNA-1 regulation on episomal vector clearance.

The vectors will be co-transfected in various combinations into iPSCs, and the transfected cells will be maintained with or without Dox for 15 passages (see Table 2 for summary of transfection conditions and expected effect). The expression of a GFP reporter will be monitored when applicable. Cell pellets will be collected at every cell passage. The status of vector clearance will be examined using qPCR vector detection screening assay at every 2-3 cell passages.

TABLE 1
List of vectors to be used for exploring the effect of EBNA-1
regulation on episomal vector clearance in iPSCs.

| Vector | | Description |
|---|---|---|
| TetOn-eGFP | TetOn regulated eGFP | Control vector for |
| | (activated by Dox) | TetOn regulation |
| TetOn-EBNA-1 | TetOn regulated EBNA-1 | Regulated EBNA-1 |
| | (activated by Dox) | vector |
| CAG-EBNA-1 | Constitutively expressed | Constitutive EBNA-1 |
| | EBNA-1 | vector |
| TetOn-EBNA-1 | TetOn regulated EBNA-1 | Regulated EBNA-1 |
| (OriP) | (activated by Dox) | vector |
| CAG-EBNA-1 | Constitutively expressed | Constitutive EBNA-1 |
| (OriP) | EBNA-1 | vector |
| SV40-eGFP | Constitutively expressed | Test vector-eGFP |
| (OriP) | eGFP | |

TABLE 2
Summary of transfection conditions and expected effect on vector clearance

| Condition | treatment | Condition purpose | Expected effect |
|---|---|---|---|
| SV40-eGFP (OriP) | N/A | Transfection | As vector is transient, fast |
| | | efficiency control | reduction of GFP |
| | | and negative control | expression is expected due |
| | | for vector retention | to fast vector clearance in |
| | | | about 2 weeks |
| SV40-eGFP (OriP): CAG- | N/A | Vector retention | With the addition of |
| EBNA-1 (OriP) | | positive control | EBNA-1 expression, |
| | | | transient vector will behave |
| | | | as an episomal vector. |
| | | | Slow reduction of GFP |
| | | | expression is expected due |
| | | | to slow vector clearance in |
| | | | >15 passages |
| TetOn-eGFP | +Dox | Tet regulation | Dox dependent expression |
| | (multiple | control | of GFP. Dox presence |
| | concentrations | | induces GFP expression. |
| | may be tested) | | Fast vector clearance as |
| | | | EBNA-1 is absent |
| TetOn-eGFP | −Dox | Tet regulation | No induction of eGFP |
| | | control | expression due to lack of |
| | | | Dox. Fast vector clearance |
| | | | as EBNA-1 is absent |
| SV40-eGFP (OriP): TetOn- | +Dox | Tet regulation of | Dox dependent expression |
| EBNA-1 | (multiple | EBNA1 expression | of EBNA-1 and |
| | concentrations | | corresponding vector |
| | may be tested) | | clearance rate (>15 |
| | | | passages for high EBNA-1 |
| | | | expression). |
| SV40-eGFP (OriP): TetOn- | −Dox | Tet regulation of | No induction of EBNA-1 |
| EBNA-1 | | EBNA1 expression | expression due to lack of |
| | | | Dox. Fast vector clearance |
| | | | in <7 passages |
| SV40-eGFP (OriP): TetOn- | +Dox | Tet regulation of | Dox dependent expression |
| EBNA-1 (OriP) | (multiple | EBNA1 expression | of EBNA-1 and |
| | concentrations | | corresponding vector |
| | may be tested) | | clearance rate (>15 |
| | | | passages for high EBNA-1 |
| | | | expression). OriP inclusion |
| | | | on EBNA-1 vector may |
| | | | further influence retention |
| | | | kinetics. |
| SV40-eGFP (OriP): TetOn- | −Dox | Tet regulation of | No induction of EBNA-1 |
| EBNA-1 (OriP) | | EBNA1 expression | expression due to lack of |
| | | | Dox. Fast vector clearance |
| | | | in <7 passages. OriP |
| | | | inclusion on EBNA-1 |
| | | | vector may further |
| | | | influence retention kinetics. |

Step 1, Experimental Goal 2: Test the Effect of Suicide Gene Incorporation on Kinetics of Vector Clearance in iPSCs

The suicide gene Thymidine kinase (TK) sequence will be cloned into the SV40-eGFP (OriP), TetOn-EBNA-1 and CAG-EBNA-1 vectors described in Table 1 (see Table 3 for list). The SV40-eGFP (OriP) vector will be used to provide transfection efficiency control and as the ‘Test’ vector. Combinations of vectors for transfection are indicated in Table 4. EBNA-1 vectors tested will initially contain OriP regions, but variants not containing OriP may also be tested (and so are included in Table 3) as appropriate.

To determine the optimal concentration of GNC to use with TK vectors, a kill curve will be constructed according to standard practices. iPSCs will be transfected with SV40-eGFP-TK (OriP) followed by treatment with various GNC concentrations, 48 hr after transfection (see Table 4 for summary of transfection conditions and expected effect). Once the optimal GNC concentration is determined, the experiment will be repeated with the EBNA-1 expression vectors, to verify its response to the optimal GNC concentration. The number of viable cells will be determined using Cell Titer-Glo luminescence cell viability assay (Promega). In addition, the number of apoptotic cells will be determined using CellEvent™ Caspase-3/7 Green Ready Probes Reagent (Invitrogen).

TABLE 3
List of vectors to be used for suicide gene activation in iPSCs.

| Vector | Description |
|---|---|
| SV40-eGFP TK (OriP) | Constitutively expressed eGFP |
| | with suicide gene (TK) |
| TetOn-EBNA-1 TK | Regulated EBNA-1 with suicide |
| | gene (TK) expressed from |
| | constitutive promoter |
| CAG-EBNA-1 TK | Constitutively expressed EBNA-1 |
| | with suicide gene (TK) |
| TetOn-EBNA-1 TK | Regulated EBNA-1 containing OriP |
| (OriP) | with suicide gene (TK) expressed |
| | from constitutive promoter |
| CAG-EBNA-1 TK | Constitutive EBNA-1 containing OriP |
| (OriP) | with suicide gene (TK) |

TABLE 4
The summary of transfection conditions and expected effect

| | | Condition | |
|---|---|---|---|
| Condition | treatment | purpose | Expected effect |
| SV40-eGFP TK (OriP) | −GNC | TK negative | Lack of suicide gene (TK) |
| | | control | substrate results in no cell |
| | | | death |
| SV40-eGFP TK (OriP): | −GNC | TK negative | Lack of suicide gene (TK) |
| CAG-EBNA-1 TK (OriP) | | control | substrate results in no cell |
| | | | death |
| SV40-eGFP TK (OriP) | +0.2 μM GNC | Identify optimal | TK substrate results (GNC) |
| SV40-eGFP TK (OriP) | +2 μM GNC | GNC | results in dose dependent |
| SV40-eGFP TK (OriP) | +20 μM GNC | concentration | induction of cell death |
| SV40-eGFP TK (OriP): | +0.2 μM GNC | Identify optimal | TK substrate results (GNC) |
| CAG-EBNA-1 TK (OriP) | | GNC | results in dose dependent |
| SV40-eGFP TK (OriP): | +2 μM GNC | concentration | induction of cell death |
| CAG-EBNA-1 TK (OriP) | | | |
| SV40-eGFP TK (OriP): | +20 μM GNC | | |
| CAG-EBNA-1 TK (OriP) | | | |
| SV40-eGFP TK (OriP): | −Dox | TK negative | Lack of suicide gene (TK) |
| TetOn-EBNA-1 TK | −GNC | control | substrate results in no cell |
| (OriP) | | | death |
| SV40-eGFP TK (OriP): | +Dox | | |
| TetOn-EBNA-1 TK | −GNC | | |
| (OriP) | | | |
| SV40-eGFP TK (OriP): | −Dox | TK activation | TK substrate results (GNC) |
| TetOn-EBNA-1 TK | With optimal | positive control | results in dose dependent |
| (OriP) | GNC | | induction of cell death. Dox |
| | concentration | | may influence vector retention |
| | as determined | | kinetics and thus influence |
| | | | dose response. |
| SV40-eGFP TK (OriP): | +Dox | | |
| TetOn-EBNA-1 TK | With optimal | | |
| (OriP) | GNC | | |
| | concentration | | |
| | as determined | | |

Step 2, Experimental Goal 1: Promote Vector Clearance in iPSC Colonies by Regulating EBNA-1 Expression Followed by Suicide Gene Activation for Vector-Free Colony Screening

For this experiment reprogramming vectors will be created by cloning Oct4, Sox2, KLF4, cMYC, Lin28, and p53DD into OriP TK vectors, in place of SV40-eGFP used in Step 1 experiments. In addition, TetOn-EBNA-1 TK vectors that were described above will be used for EBNA-1 regulation and suicide gene activation (see Table 5 for the list and description of vectors used for cellular reprogramming and vector clearance induction). To induce cellular reprogramming, PBMCs will be co-nucleofected with Oct4-TK (OriP), Sox2/KLF4 TK (OriP), cMYC/Lin28 TK (OriP), mp53DD TK (OriP) and TetOn-EBNA-1 TK vectors.

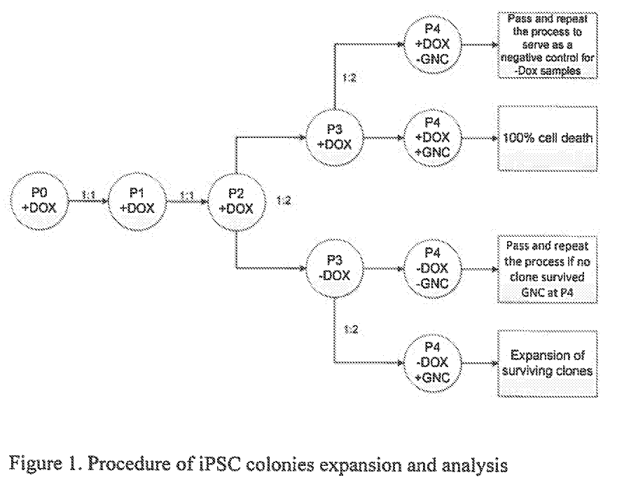

Nucleofected cells will be plated onto P0 plate and cultured as described in reprogramming protocol (see Appendix A for the reprogramming protocol) and cultured in the presence of Dox to allow EBNA-1 expression and the retention of the reprogramming vectors. Colonies that appear on P0 plate will be manually passed into separate wells and fed with culture medium supplemented with Dox. P1 colonies of each clone will be also passed at 1:1 ratio and fed with culture medium supplemented with Dox. P2 colonies of each clone will be passed at 1:2 ratio to two wells and fed with culture medium supplemented with Dox. P3 colonies of each clone from two wells will be passed at 1:2 ratio to generate four replica wells. Two wells of each iPSC clone will be maintained in culture medium supplemented with Dox, while the other two will be cultured in medium without Dox. At P4, to induce cell death of the colonies that still retain the vector, GNC will be added to the culture medium of two wells of each iPSC clone—one treated with Dox and one that is not treated with Dox. Cell death should be observed in all colonies that are cultured with Dox. Surviving clones, from the wells that were not treated with Dox will be further expanded. If no clone has survived GNC treatment at P4, the remaining replica wells will be passed to P5 and the process will be repeated till vector-free clones are identified. During the expansion process cell pellets will be collected and analyzed using qPCR vector detection screening assay to confirm vector clearance (See FIG. 1 depicting the procedure of iPSC colonies expansion and analysis). As a positive control for the reprogramming process and the ability of the given somatic cells to be reprogrammed, the cells will be nucleofected with Okita reprogramming set Okita, K. et al. (2013). “An efficient nonviral method to generate integration-free human-induced pluripotent stem cells from cord blood and peripheral blood cells.” Stem Cells 31(3): 458-66).

TABLE 5
List of vectors to be used for cellular reprogramming
and induction of vector clearance followed by
screening with suicide gene activation.

| Vector | Description |
|---|---|
| Oct4-TK (OriP) | Constitutively expressed Oct4, |
| | reprogramming vector |
| Sox2/KLF4 TK (OriP) | Constitutively active Sox2 and KLF4, |
| | reprogramming vector |
| cMYC/Lin28 TK (OriP) | Constitutively active cMYC and Lin28, |
| | reprogramming vector |
| mp53DD TK (OriP) | Constitutively active p53, reprogramming |
| | vector |
| TetOn-EBNA-1 TK | TetOn regulated EBNA-1 (activated by |
| | Dox) and constitutively |
| | expressed suicide gene (TK) |
| TetOn-EBNA-1 TK (OriP) | TetOn regulated EBNA-1 (activated by |
| | Dox) and constitutively |
| | expressed suicide gene (TK) |

### Step 2, Experimental Goal 2: Determine the Feasibility of Inducing Vector Clearance at P0 Plate by Regulating EBNA-1 Expression Followed by Suicide Gene Activation

Cellular reprogramming relies on the ability to control expression of reprogramming factors in somatic cells both in terms of absolute expression levels and temporally. It is widely anticipated that current methods are sub-optimal in both respects.

The timing of removing EBNA-1 induction (by removing Dox from media) to induce vector clearance could potentially negatively affect the reprogramming efficiency. In order to be able to pick iPSC colonies from the P0 plate, as a pool instead of individual colonies, the optimal time point for Dox withdraw, without affecting the reprogramming efficiency, needs to be determined. For this experiment PBMCs will be co-nucleofected with Oct4 TK (OriP), Sox2/KLF4 TK (OriP), cMYC/Lin28 TK (OriP), mp53DD TK (OriP) and TetOn-EBNA1 TK vectors, and cultured as described in the reprogramming protocol (see Appendix A for the reprogramming protocol) in the presence of Dox to allow EBNA-1 expression and the retention of the reprogramming vectors.

Dox will be withdrawn from the culture medium at various time points after nucleofection as shown in Table 6. Appearance of iPSC colonies under the various experimental conditions will be monitored using a phase microscope. When colonies are large enough to subculture, GNC will be added to the culture medium to activate TK and select for survival of vector-free colonies. Surviving colonies will be further picked and expanded. During the expansion process cell pellets will be collected and analyzed using a qPCR vector detection screening assay to confirm vector clearance. As a positive control for the reprogramming process and the ability of the given somatic cells to be reprogrammed, the cells will be nucleofected with Okita reprogramming set (Okita, Yamakawa et al. 2013).

TABLE 6
Experimental conditions and Dox removal schedule on P0 plate

| | Well 1-2 | Well 3-4 | Well 5-6 | Well 7-8 |
|---|---|---|---|---|
| Day 0-8 | +Dox | +Dox | +Dox | −Dox |
| Day 8-16 | +Dox | +Dox | −Dox | −Dox |
| Day 16-24 | +Dox | −Dox | −Dox | −Dox |
| Day 24-28 | +Dox/+GNC | −Dox/+GNC | −Dox/+GNC | −Dox/+GNC |

| | | | |
|---|---|---|---|
| Condition | Test for new | Test Dox withraw effect on | Negative contol for |
| purpose | reprogramming set | reprogramming efficiency | reprogramming |

Supporting Data: Cytotoxic Effect of the Suicide Gene Substrate on hPSCs that do not Express the Suicide Gene.

Since we propose to activate a suicide gene in somatic cells during or after cellular reprogramming, the cytotoxic effect of ganciclovir and 5-FC on hiPSC that do not express the respective suicide gene was tested. hiPSCs were cultured for 48 hours in the presence of ganciclovir at a final concentration of 0.2, 2 or 20 μM and 5-FC at a final concentration of 1, 10 and 100 μM were added to hiPSCs. Cell were fixed and stained for alkaline phosphatase activity after 48 hr. Staining results show no cytotoxic effect of ganciclovir or 5-FC on hiPSCs. Additional experiments will determine the required levels of ganciclovir and 5-FC in hiPSCs that express the suicide gene, to determine the minimum concentration required.

Positive Effect of Doxycycline on hPSCs

EBNA-1 regulation could be potentially achieved by using doxycycline inducible promoter system. A recent publication has reported that doxycycline exerts dramatic effects on hPSCs survival and self-renewal (Chang, M. Y. et al. “Doxycycline enhances survival and self-renewal of human pluripotent stem cells.” Stem Cell Reports 3(2):353-64 (2014)). Doxycycline effects are not associated with its antibacterial action, but mediated by direct activation of a PI3K-AKT intracellular signal. These findings indicate doxycycline as a useful supplement for stem cell cultures, facilitating their growth and maintenance, and therefore no negative effects of using it to regulate EBNA-1 expression are anticipated.

1. A method of producing induced human pluripotent stem cells (iPSCs) that are essentially free of reprogramming vector(s), comprising:
(a) introducing the reprogramming vector(s) into a human somatic cell to produce a first cell population, wherein the reprogramming vector(s) comprises a viral origin of replication, an expression cassette encoding an iPSC reprogramming factor, a synthetic transcription factor, or both, and a suicide gene;
(b) culturing the first cell population to effect expression of the reprogramming factor, the synthetic transcription factor, or both, to produce a second cell population having traits consistent with embryonic stem cells;
(c) contacting the second cell population with a suicide gene substrate to produce iPSCs that are essentially free of reprogramming vector(s).

2. The method of claim 1, wherein the suicide gene is selected from the group consisting of thymidine kinase and cytosine deaminase.

3. The method of claim 1, wherein the origin of replication is OriP.

4. The method of claim 1, wherein the expression cassette comprises a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1.

5. The method of, wherein the somatic cell is selected from the group consisting of human peripheral blood mononuclear cells, fibroblasts, keratinocytes, hematopoietic cells, mesenchymal cells, liver cells, stomach cells and β cells.

6. The method of claim 1, wherein the iPSC reprogramming factor is selected from the group consisting of one or more of Sox-2, Oct-4, Nanog, KLF4, cMYC, Lin-28, and p53DD.

7. (canceled)

8. (canceled)

9. The method of claim 1, further comprising: (d) screening the cell population of (c) for the presence of the episomal reprogramming vector.

10. The method of claim 9, further comprising: (e) culturing the screened cells of (d) that do not contain the episomal reprogramming vector.

11. (canceled)

12. The method of claim 1, further comprising after step (b), but before step (c), subculturing cells of the second cell population.

13. (canceled)

14. A method of producing induced human pluripotent stem cells (iPSCs) that are essentially free of episomal reprogramming vector(s), comprising:
(a) introducing an episomal reprogramming vector(s) into a somatic cell to produce a first cell population, wherein the episomal reprogramming vector(s) comprise
(i) an OriP replication origin,
(ii) an expression cassette encoding an iPSC reprogramming factor, a synthetic transcription factor, or both,
(iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328, and
(iv) a thymidine kinase or cytosine deaminase suicide gene;
(b) culturing the first cell population to effect expression of the reprogramming factor, the synthetic transcription factor, or both, to produce a second cell population having traits consistent with embryonic stem cells;
(c) contacting the second cell population with a suicide gene substrate to produce iPSCs that are essentially free of an episomal reprogramming vector.

15. (canceled)

16. (canceled)

17. (canceled)

18. (canceled)

19. A method of producing induced human pluripotent stem cells (iPSCs) that are essentially free of a reprogramming vector, comprising:
(a) introducing a reprogramming vector into a somatic cell to produce a first cell population, wherein the reprogramming vector comprises (i) a viral origin of replication, (ii) an expression cassette encoding an iPSC reprogramming factor, a synthetic transcription factor, or both, (iii) a gene regulating extrachromosomal replication and partitioning of the reprogramming vector, and (iv) a regulated promoter system;
(b) culturing the first cell population to effect expression of the reprogramming factors, the synthetic transcription factor, or both, to produce a second cell population having traits consistent with embryonic stem cells, wherein during culture of the first cell population the episomal reprogramming vector is replicated;
(c) culturing the second cell population wherein the gene regulating extrachromosomal replication and partitioning of the reprogramming vector is regulated such that the reprogramming vector is lost during cell division to produce iPSCs that are essentially free of the reprogramming vector.

20. The method of claim 19, wherein the gene regulating transcription of the episomal reprogramming vector comprises a tetracycline or tetracycline derivative activated system.

21. (canceled)

22. (canceled)

23. (canceled)

24. (canceled)

25. (canceled)

26. (canceled)

27. (canceled)

28. (canceled)

29. The method of claim 19, further comprising: (d) screening the cell population of (c) for the presence of the episomal reprogramming vector.

30. The method of claim 29, further comprising: (e) culturing the screened cells of (d) that do not contain the episomal reprogramming vector.

31. The method of claim 29, wherein the screening comprises a qPCR vector detection assay.

32. (canceled)

33. (canceled)

34. A method of producing induced human pluripotent stem cells (iPSCs) that are essentially free of an episomal reprogramming vector, comprising:
(a) introducing an episomal reprogramming vector into a somatic cell to produce a first cell population, wherein the episomal reprogramming vector comprises
(i) an OriP replication origin,
(ii) an expression cassette encoding an iPSC reprogramming factor, a synthetic transcription factor, or both,
(iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65 to 328 of EBNA-1, and
(iv) a tetracycline or tetracycline derivative regulated promoter system (TetOn or TetOff);
(b) culturing the first cell population to produce a second cell population having traits consistent with embryonic stem cells, wherein during culturing the episomal reprogramming vector is replicated;
(c) culturing the second cell population to produce a third cell population, wherein during culturing the episomal reprogramming vector is not replicated;
(d) selecting colonies from the third cell population to produce a fourth cell population; and
(e) culturing the fourth cell population to produce iPSCs that are essentially free of the episomal reprogramming vector.

35. (canceled)

36. (canceled)

37. (canceled)

38. (canceled)

39. A method of producing induced human pluripotent stem cells (iPSCs) that are essentially free of a reprogramming vector, comprising:
(a) introducing a reprogramming vector into a somatic cell to produce a first cell population, wherein the reprogramming vector comprises (i) a viral origin of replication, (ii) an expression cassette encoding an iPSC reprogramming factor, a synthetic transcription factor, or both, (iii) a gene regulating extrachromosomal replication and partitioning of the reprogramming vector, (iv) a regulated promoter system and (v) a suicide gene;
(b) culturing the first cell population to effect expression of the reprogramming factor, the synthetic transcription factor, or both, to produce a second cell population having traits consistent with embryonic stem cells, wherein during culture the reprogramming vector is replicated;
(c) culturing the second cell population wherein the gene regulating extrachromosomal replication and partitioning is regulated such that the reprogramming vector is lost during cell division, to produce a third cell population comprising iPSCs that are substantially free of the reprogramming vector;
(d) contacting the third cell population with a suicide gene substrate to produce iPSCs that are essentially free of the reprogramming vector.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

50. (canceled)

51. (canceled)

52. A method of producing induced human pluripotent stem cells (iPSCs) that are essentially free of an episomal reprogramming vector, comprising:
(a) introducing an episomal reprogramming vector into a somatic cell to produce a first cell population, wherein the episomal reprogramming vector comprises:
(i) an OriP replication origin,
(ii) an expression cassette encoding an iPSC reprogramming factor, a synthetic transcription factor, or both,
(iii) a polynucleotide encoding EBNA-1 of EBV, a derivative of EBNA-1 that has a deletion of residues 65 to 89 of EBNA-1, a derivative of EBNA-1 that has a deletion of residues 90 to 328 of EBNA-1, or or a derivative of EBNA-1 that has a deletion of residues 65-328 of EBNA-1,
(iv) a tetracycline or derivative regulated promoter system (TetOn or TetOff), and
(v) a thymidine kinase or cytosine deaminase suicide gene;
(b) culturing the first cell population to effect expression of the reprogramming factors to produce a second cell population having traits consistent with embryonic stem cells, wherein during culturing the episomal reprogramming vector is replicated;
(c) culturing the second cell population to produce a third cell population comprising iPSCs that are substantially free of the episomal reprogramming vector, wherein during culturing of the second cell population, the episomal reprogramming vector is not replicated;
(d) contacting the third cell population with a suicide gene substrate to produce iPSCs that are essentially free of the episomal reprogramming vector.

53. (canceled)

54. (canceled)

55. (canceled)

56. An episomal reprogramming vector comprising:
(a) an OriP origin of replication;
(b) an expression cassette encoding an iPSC reprogramming factor, a synthetic transcription factor, or both, and
(c) a polynucleotide molecule encoding EBNA-1 of EBV, a derivative of EBNA-1 that has a deletion of residues 65 to 89 of EBNA-1, a derivative of EBNA-1 that has a deletion of residues 90 to 328 of EBNA-1, or a derivative of EBNA-1 that has a deletion of residues 65-328 of EBNA-1; and element selected from
(d1) a suicide gene;
(d2) a thymidine kinase or cytosine deaminase suicide gene
(d3) a regulated promoter system;
(d4) a TetOn or TetOff system;
(d5) a TetOn or TetOff system; and a suicide gene; and/or
(d6) a TetOn or TetOff system, and a thymidine kinase or cytosine deaminase suicide gene.

57. (canceled)

58. (canceled)

59. (canceled)

60. (canceled)

61. (canceled)

62. The vector of any one of claim 56, wherein the iPSC reprogramming factor is selected from the group consisting of one or more of Sox-2, Oct-4, Nanog, KLF4, cMYC, Lin-28, and p53DD.

63. (canceled)

64. (canceled)
